(19) United States
(12) Patent Application Publication
(10) Pub. No.: US 2021/0261971 A1
(43) Pub. Date: Aug. 26, 2021

(54) COMPOSITIONS AND METHODS FOR AMELIORATING TISSUE INJURY, ENHANCING LIVER REGENERATION AND STEM CELL THERAPIES

(71) Applicant: UNIVERSITY OF SOUTHERN CALIFORNIA, Los Angeles, CA (US)

(72) Inventor: Laurie D. DE LEVE, Los Angeles, CA (US)

(21) Appl. No.: 17/254,194

(22) PCT Filed: Jun. 25, 2019
(86) PCT No.: PCT/US2019/038912

§ 371 Date: Dec. 18, 2020

Related U.S. Application Data
(60) Provisional application No. 62/689,373, filed on Jun. 25, 2018.

(51) Int. Cl. C12N 15/113; A61P 9/10; A61P 1/16

(52) U.S. Cl. CPC C12N 15/1137; C12N 2310/11; A61P 1/16; A61P 9/10

(57) ABSTRACT

In alternative embodiments, provided are compositions, including pharmaceutical compositions and formulations, products of manufacture and kits, and methods, for: enhancing or accelerating liver regeneration, optionally enhancing or accelerating liver regeneration after tissue injury or liver resection; enhancing or accelerating tissue repair, optionally enhancing or accelerating tissue repair after a trauma, an injury or an infection, wherein optionally the injury is an ischemia-reperfusion injury comprising: administering to an individual in need thereof, a compound or composition capable of inhibiting or decreasing the expression or activity of a matrix metalloproteinase (MMP) in a tissue-specific or tissue-selective manner, in in an end organ specific manner, or administering to the organ, for example, a liver, of an individual in need thereof a compound or composition capable of inhibiting or decreasing the expression or activity of a matrix metallo-proteinase.

### RELATED APPLICATIONS

This Patent Convention Treaty (PCT) International Application claims the benefit of priority to U.S. Provisional Application No. 62/689,373, Jun. 25, 2018. The aforementioned application is expressly incorporated herein by reference in its entirety and for all purposes.

### STATEMENT AS TO FEDERALLY SPONSORED RESEARCH

This invention was made with government support under grant no. DK 46357, awarded by NIH/NIDDK (National Institute of Diabetes and Digestive and Kidney Diseases). The government has certain rights in the invention.

### TECHNICAL FIELD

This invention generally relates to stem cell therapy, tissue repair after injury and tissue regeneration. In alternative embodiments, provided are compositions, including pharmaceutical compositions and formulations, products of manufacture and kits, and methods, for: enhancing or accelerating liver regeneration, optionally enhancing or accelerating liver regeneration after tissue injury or liver resection; enhancing or accelerating organ or tissue, e.g., heart, brain, lung, pancreas, kidney, muscle, bone, skin, trachea, arterial or venous blood vessels, intestine, spinal cord, nerve, brain, skin or liver repair, optionally enhancing or accelerating tissue repair after a trauma, an injury or an infection, wherein optionally the injury is an ischemia-reperfusion injury, e.g., a heart attack or a stroke; or, reducing the extent of or abolishing ischemia-reperfusion injury in a normal organ, e.g., a liver, or a fatty liver, or a cadaver or donor organ, e.g., a liver, or a transplant organ, e.g., a liver, comprising: administering to an individual in need thereof, a compound or composition capable of inhibiting or decreasing the expression or activity of a matrix metalloproteinase (MMP) in a tissue-specific or tissue-selective manner, in an end organ specific manner, or administering to the organ (e.g., a liver) of an individual in need thereof a compound or composition capable of inhibiting or decreasing the expression or activity of a matrix metallo-proteinase (MMP). In alternative embodiments, a compound or composition capable of inhibiting or decreasing the expression or activity of a matrix metalloproteinase (MMP) is administered to a donor organ (or the donor organ is treated with the compound or composition capable of inhibiting or decreasing the expression or activity of the MMP), including a donor organ such as a liver, kidney or heart, and optionally the donor organ is treated in a perfusion bath. In alternative embodiments, provided are compositions, including pharmaceutical compositions and formulations, products of manufacture and kits, and methods, for: enhancing or accelerating engraftment of exogenous stem cells, which can be used for stem cell therapy.

### BACKGROUND

In liver injury, recruitment of bone marrow endothelial progenitor cells of liver sinusoidal endothelial cells (also called “sprocs”) is necessary for normal liver regeneration. Recruitment of BM endothelial progenitor cells promotes recovery after liver injury and promotes liver regeneration.

Hepatic VEGF-sdf1 signaling is necessary to recruit bone marrow (BM) endothelial progenitor cells to the liver. Hepatic vascular endothelial growth factor (VEGF) is a central regulator of the endothelial progenitor cell recruitment (to the liver) process, and stromal cell-derived factor-1 (also called sdf-1 or CXCL-12) acts downstream from VEGF to mediate recruitment of bone marrow endothelial progenitor cells.

Matrix metalloproteinases (MMPs), also known as matrixins, are calcium-dependent zinc-containing endopeptidases. MMP-1, MMP-2, MMP-3, MMP-11, and MMP-13 are among the MMPs constitutively expressed in organs, including livers. Systemic MMP inhibition has been tried to limit liver injury, but with minimal benefit. Because systemic MMP inhibition prevents endothelial progenitor cells from leaving the bone marrow, systemic inhibition has minimal benefit or is detrimental. Systemic inhibition of MMP had minimal benefit in protecting small for size injury (in living donor transplantation or in split cadaver donor transplantation, a portion of the liver is transplanted, and given that the circulation is meant for a larger organ, the grafted liver is damaged).

### SUMMARY

In alternative embodiments, provided are methods for:

(i) enhancing or accelerating a liver regeneration, optionally enhancing or accelerating liver regeneration after tissue injury, toxic liver injury, acute liver failure, liver transplantation, living-donor related liver transplantation, or liver resection,

(ii) enhancing or accelerating repair of an organ, e.g., a liver repair, optionally enhancing or accelerating repair of an organ, e.g., a liver repair after a trauma, an injury or an infection, wherein optionally the injury is an ischemia-reperfusion injury, optionally a heart attack or a stroke,

(iii) reducing the extent of or abolishing ischemia-reperfusion injury in an organ, e.g., in a normal liver or a fatty liver, a brain, lung, pancreas, kidney or a heart, or in a cadaver or a donor organ, e.g., in a liver or a transplant liver or a heart or a lung, pancreas, kidney or transplant heart, or

(iv) enhancing bone marrow endothelial progenitor cell mobilization and engraftment in an organ, e.g., a heart, a brain or a liver, or enhancing chemoattraction of bone marrow progenitor cells to the organ, e.g., a heart, a brain, lung, pancreas, kidney, muscle, bone, skin, trachea, arterial or venous blood vessels, intestine, spinal cord, nerve, or a liver,

wherein optionally the bone marrow progenitor cell comprises a bone marrow progenitor of liver sinusoidal endothelial cell (a so-called “sproc”);

comprising:

(a) (i) administering to an individual in need thereof, or to an organ or a tissue, e.g., a liver, a heart or a brain, in need thereof if the organ or tissue is a transplant organ or tissue or a cadaver or donor organ or tissue intended for transplant, a compound or composition capable of inhibiting or decreasing the expression or activity of a matrix metalloproteinase (MMP) in an organ-specific or an organ-selective manner, or

(ii) administering to an organ or tissue, (wherein optionally the organ or tissue is a liver, a heart, a kidney, muscle, bone, skin, trachea, arterial or venous blood vessels, intestine, nerve or a brain) of an individual in need thereof, or administering to or treating the organ in need thereof if the organ or tissue is a transplant organ, a cadaver or a donor organ or tissue intended for transplant or study, a compound or composition capable of inhibiting or decreasing the expression or activity of a matrix metalloproteinase (MMP),

wherein the compound or composition acts in an organ-specific or an organ-selective manner if the compound or composition is administered systemically,

and optionally the organ or tissue is treated with the compound or composition capable of inhibiting or decreasing the expression or activity of the MMP in a perfusion bath; or

(b) (1) providing a compound or composition capable of inhibiting or decreasing the expression or activity of a matrix metalloproteinase (MMP) in a liver-specific or liver-selective manner,

wherein optionally the matrix metalloproteinase (MMP) is a matrix metalloproteinase-9 (MMP-9), and optionally the MMP inhibition is MMP-9 specific,

and optionally the MMP is MMP-1, MMP-2, MMP-3, MMP-11, or MMP-13, and optionally the MMP inhibition is MMP-1, MMP-2, MMP-3, MMP-11, or MMP-13 specific; and

(2) administering the compound or composition to the individual in need thereof if the compound or composition acts in an organ-specific manner, or contacting the organ or tissue with the compound or composition,

wherein optionally the organ is a transplant organ or a cadaver or donor organ or tissue intended for transplant, and optionally the compound or composition is administered to the organ before removal of the organ or tissue from the cadaver or donor,

(i) enhancing or accelerating a liver regeneration, optionally enhancing or accelerating liver regeneration after tissue injury, toxic liver injury, acute liver failure, liver transplantation, living-donor related liver transplantation, or liver resection,

(ii) enhancing or accelerating repair of an organ or tissue, e.g., a liver repair, optionally enhancing or accelerating repair of an organ or tissue, e.g., a liver repair after a trauma, an injury or an infection, wherein optionally the injury is an ischemia-reperfusion injury, optionally a heart attack or a stroke,

(iii) reducing the extent of or abolishing ischemia-reperfusion injury in an organ or tissue, e.g., in a normal liver or a fatty liver, a brain, lung, pancreas, kidney or a heart, or in a cadaver or a donor organ or tissue, e.g., in a liver or a transplant liver or a heart or a lung, pancreas, kidney or transplant heart, or

(iv) enhancing bone marrow endothelial progenitor cell mobilization and engraftment in an organ or tissue, e.g., a heart, a brain or a liver, or enhancing chemoattraction of bone marrow progenitor cells to the organ or tissue, e.g., a heart, a brain, lung, pancreas, kidney, muscle, bone, skin, trachea, arterial or venous blood vessels, intestine, spinal cord, or a liver.

In alternative embodiments of methods as provided herein:

(a) the compound or composition capable of inhibiting or decreasing the expression or activity of the MMP protein, transcript and/or gene, optionally in a liver-selective manner, is or comprises:
(1) a nucleic acid, and optionally the nucleic acid is an inhibitory nucleic acid comprising: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme capable of capable of inhibiting or decreasing the expression or activity of the MMP protein, transcript and/or gene,
(2) a peptide or polypeptide, wherein optionally the polypeptide is or comprises an antibody or fragment thereof or equivalent thereof, capable of specifically binding the MMP, and is capable of inhibiting or decreasing the activity of the MMP enzyme, transcript and/or gene, or
(3) a small molecule, lipid, saccharide, nucleic acid or polysaccharide capable of inhibiting or decreasing the activity of the MMP enzyme, transcript and/or gene,
wherein optionally the small molecule comprises prinomastat, marimastat, batimastat, cipemastat, ilomastat (also known as galardin), rebimastat, tanomastat or any combination thereof,

(b) the compound or composition is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally;

wherein optionally the formulation or pharmaceutical composition is contained in or carried in a nanoparticle, a particle, a micelle or a liposome or lipoplex, a polymersome, a polyplex or a dendrimer; or

(c) the compound or composition, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant.

In alternative embodiments, the nucleic acid capable of inhibiting or decreasing the expression or activity of the MMP enzyme, transcript and/or gene comprises or is contained in a nucleic acid construct or a chimeric or a recombinant nucleic acid, or an expression cassette, vector, plasmid, phagemid or artificial chromosome, optionally stably integrated into the cell's chromosome, or optionally stably episomally expressed, and optionally the cell is a liver cell or a cell in a liver tissue or organ.

In alternative embodiments, the cell or the liver cell is a mammalian cell, wherein optionally the mammalian cell is an animal or a human cell, or a brain, a lung, a pancreas, a kidney or a heart cell.

In alternative embodiments, provided are kits comprising a compound or composition or a formulation or a pharmaceutical composition as provided herein, and optionally comprising instructions on practicing a method as provided herein.

In alternative embodiments, provided are Uses of a compound or composition or a formulation as provided herein, in the manufacture of a medicament.

In alternative embodiments, provided are Uses of a compound or composition, or a formulation or a pharmaceutical composition as provided herein in the manufacture of a medicament for:

(i) enhancing or accelerating a liver regeneration, optionally enhancing or accelerating liver regeneration after tissue injury, toxic liver injury, acute liver failure, liver transplantation, living-donor related liver transplantation, or liver resection,

(ii) enhancing or accelerating repair of an organ or tissue, e.g., a liver repair, optionally enhancing or accelerating repair of an organ or tissue, e.g., a liver repair after a trauma, an injury or an infection, wherein optionally the injury is an ischemia-reperfusion injury, optionally a heart attack or a stroke,

(iii) reducing the extent of or abolishing ischemia-reperfusion injury in an organ or tissue, e.g., in a normal liver or a fatty liver, a brain, lung, pancreas, kidney or a heart, or in a cadaver or a donor organ or tissue, e.g., in a liver or a transplant liver or a heart or a lung, pancreas, kidney or transplant heart, or

(iv) enhancing bone marrow endothelial progenitor cell mobilization and engraftment in an organ or tissue, e.g., a heart, a brain or a liver, muscle, bone, skin, trachea, arterial or venous blood vessels, intestine, spinal cord, or enhancing chemoattraction of bone marrow progenitor cells to the organ or tissue, e.g., a heart, a brain, lung, pancreas, kidney, muscle, bone, skin, trachea, arterial or venous blood vessels, intestine, spinal cord, or a liver.

In alternative embodiments, provided are compounds or compositions or formulations as used in methods as provided herein, for use in:

(i) enhancing or accelerating a liver regeneration, optionally enhancing or accelerating liver regeneration after tissue injury, toxic liver injury, acute liver failure, liver transplantation, living-donor related liver transplantation, or liver resection,

(ii) enhancing or accelerating repair of an organ, e.g., a liver repair, optionally enhancing or accelerating repair of an organ, e.g., a liver repair after a trauma, an injury or an infection, wherein optionally the injury is an ischemia-reperfusion injury, optionally a heart attack or a stroke,

(iii) reducing the extent of or abolishing ischemia-reperfusion injury in an organ, e.g., in a normal liver or a fatty liver, a brain, lung, pancreas, kidney or a heart, or in a cadaver or a donor organ, e.g., in a liver or a transplant liver or a heart or a lung, pancreas, kidney or transplant heart, or

(iv) enhancing bone marrow endothelial progenitor cell mobilization and engraftment in an organ or tissue, e.g., a heart, a brain or a liver, or enhancing chemoattraction of bone marrow progenitor cells to the organ or tissue, e.g., a heart, a brain, lung, pancreas, kidney, muscle, bone, skin, trachea, arterial or venous blood vessels, intestine, spinal cord, or a liver;

wherein optionally the use comprises a method for administering to an individual in need thereof, or contacting a liver cell or liver tissue or organ, with, a compound or composition capable of inhibiting or decreasing the expression or activity of an MMP enzyme, transcript and/or gene.

In alternative embodiments, provided are methods for:

(i) enhancing infused or exogenous progenitor cell, optionally bone marrow endothelial progenitor cell, engraftment in an organ or a tissue (e.g., an “end organ or tissue”), wherein optionally the organ or tissue is a heart, kidney, brain, spinal cord, lung, or a liver, by preventing or inhibiting proteolytic cleavage of a sdf1 in the organ or the tissue (e.g., the “end organ”), thereby preserving the chemoattractant effect of the sdf1 and also preserving a bone marrow sdf1, thereby reducing release of an endogenous progenitor cell, optionally an endogenous bone marrow endothelial progenitor cell, from the bone marrow, to decrease competition for engraftment of the infused or exogenous progenitor cell by the endogenous progenitor cell,

(ii) reducing release of endogenous progenitor cells, optionally sprocs, from the bone marrow, and/or

(iii) preventing or inhibiting proteolytic cleavage of the sdf1 systemically; comprising:

(a) administering to an individual in need thereof a compound or composition capable of inhibiting or decreasing the expression or activity of a matrix metalloproteinase (MMP) in a systemic manner, or treating the organ or tissue with the compound or composition capable of inhibiting or decreasing the expression or activity of the MMP, and optionally the treatment is in a perfusion bath; or

(b) (1) providing a compound or composition capable of inhibiting or decreasing the expression or activity of a matrix metalloproteinase (MMP) in a systemic manner, wherein optionally the matrix metalloproteinase (MMP) is a matrix metalloproteinase-9 (MMP-9), and optionally the MMP inhibition is MMP-9 specific, and optionally the MMP is MMP-1, MMP-2, MMP-3, MMP-11, or MMP-13, and optionally the MMP inhibition is MMP-1, MMP-2, MMP-3, MMP-11, or MMP-13 specific; and

(2) administering the compound or composition to the individual in need thereof, thereby

(i) enhancing infused or exogenous progenitor cell, optionally bone marrow endothelial progenitor cell, engraftment in an organ or a tissue (e.g., an “end organ or tissue”), wherein optionally the organ or tissue is a heart, kidney, brain, spinal cord, lung, or a liver, by preventing or inhibiting proteolytic cleavage of a sdf1 in the organ or the tissue (e.g., the “end organ”), thereby preserving the chemoattractant effect of the sdf1 and also preserving a bone marrow sdf1, thereby reducing release of an endogenous progenitor cell, optionally an endogenous bone marrow endothelial progenitor cell, from the bone marrow, to decrease competition for engraftment of the infused or exogenous progenitor cell by the endogenous progenitor cell,

(ii) reducing release of endogenous progenitor cells, optionally sprocs, from the bone marrow, and/or

(iii) preventing or inhibiting proteolytic cleavage of the sdf1 systemically.

In alternative embodiments of methods as provided herein:

(a) the compound or composition capable of inhibiting or decreasing the expression or activity of the MMP protein, transcript and/or gene, optionally in an organ-selective manner, is or comprises:
(1) a nucleic acid, and optionally the nucleic acid is an inhibitory nucleic acid comprising: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme capable of capable of inhibiting or decreasing the expression or activity of the MMP protein, transcript and/or gene,
(2) a peptide or polypeptide, wherein optionally the polypeptide is or comprises an antibody or fragment thereof or equivalent thereof, capable of specifically binding the MMP, and is capable of inhibiting or decreasing the activity of the MMP enzyme, transcript and/or gene, or
(3) a small molecule, lipid, saccharide, nucleic acid or polysaccharide capable of inhibiting or decreasing the activity of the MMP enzyme, transcript and/or gene,
   wherein optionally the small molecule comprises prinomastat, marimastat, batimastat, cipemastat, ilomastat (also known as galardin), rebimastat, tanomastat or any combination thereof,

(b) the compound or composition is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally;

wherein optionally the formulation or pharmaceutical composition is contained in or carried in a nanoparticle, a particle, a micelle or a liposome or lipoplex, a polymersome, a polyplex or a dendrimer; or

(c) the compound or composition, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant.

In alternative embodiments, the nucleic acid capable of inhibiting or decreasing the expression or activity of the MMP enzyme, transcript and/or gene comprises or is contained in a nucleic acid construct or a chimeric or a recombinant nucleic acid, or an expression cassette, vector, plasmid, phagemid or artificial chromosome, optionally stably integrated into the cell's chromosome, or optionally stably episomally expressed, and optionally the cell is a liver cell or a cell in a liver tissue or organ.

In alternative embodiments, the cell or the liver cell is a mammalian cell, wherein optionally the mammalian cell is an animal or a human cell, or a brain, a lung, a pancreas, a kidney or a heart cell.

In alternative embodiments, provided are Uses of a compound or composition, or a formulation or a pharmaceutical composition used in a method as provided herein in the manufacture of a medicament for:

(i) enhancing infused or exogenous progenitor cell, optionally bone marrow endothelial progenitor cell, engraftment in an organ or a tissue (e.g., an “end organ or tissue”), wherein optionally the organ or tissue is a heart, kidney, brain, spinal cord, lung, or a liver, by preventing or inhibiting proteolytic cleavage of a sdf1 in the organ or the tissue (e.g., the “end organ”), thereby preserving the chemoattractant effect of the sdf1 and also preserving a bone marrow sdf1, thereby reducing release of an endogenous progenitor cell, optionally an endogenous bone marrow endothelial progenitor cell, from the bone marrow, to decrease competition for engraftment of the infused or exogenous progenitor cell by the endogenous progenitor cell,

(ii) reducing release of endogenous progenitor cells, optionally sprocs, from the bone marrow, and/or

(iii) preventing or inhibiting proteolytic cleavage of the sdf1 systemically.

In alternative embodiments, provided are compounds or compositions or formulations as provided herein, for use in:

(i) enhancing infused or exogenous progenitor cell, optionally bone marrow endothelial progenitor cell, engraftment in an organ or a tissue (e.g., an “end organ or tissue”), wherein optionally the organ or tissue is a heart, kidney, brain, spinal cord, lung, or a liver, by preventing or inhibiting proteolytic cleavage of a sdf1 in the organ or the tissue (e.g., the “end organ”), thereby preserving the chemoattractant effect of the sdf1 and also preserving a bone marrow sdf1, thereby reducing release of an endogenous progenitor cell, optionally an endogenous bone marrow endothelial progenitor cell, from the bone marrow, to decrease competition for engraftment of the infused or exogenous progenitor cell by the endogenous progenitor cell,

(ii) reducing release of endogenous progenitor cells, optionally sprocs, from the bone marrow, and/or

(iii) preventing or inhibiting proteolytic cleavage of the sdf1 systemically, wherein optionally the use comprises a method for administering to an individual in need thereof a compound or composition capable of systemically inhibiting or decreasing the expression or activity of an MMP enzyme, transcript and/or gene.

The details of one or more exemplary embodiments as provided herein are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

Figures are described in detail herein:

FIG. 1 schematically illustrates the pros and cons of proteolytic cleavage by MMP-9, where in injury, VEGF-sdf-1 increases and attracts bone marrow (BM) sprocs (which is a beneficial effect); but that in liver injury MMP-9 also increases and can cleave both VEGF and sdf-1, which prevents the ability to promote recruitment and engraftment of BM sprocs. Sdf-1 anchors BM cells, so that MMP cleavage is necessary to release BM sprocs (sprocs=endothelial progenitor cells specific for sinusoidal endothelial cells) to repair endothelial cell loss. Thus, MMP activity in the BM is a wanted effect (to allow release of BM sprocs) and MMP activity in the liver is detrimental because it reduces chemoattraction of BM sprocs.

FIG. 2 graphically illustrates that antisense oligonucleotides (ASO) that inhibit liver matrix metalloproteinase-9 (MMP-9) prevent liver ischemia-reperfusion injury, where the data shows that levels of alanine aminotransferase (ALT) and aspartate aminotransferase (AST), which are indicators of liver injury, were elevated in the control ASO group and dramatically decreased (virtually down to normal control levels) when the liver cells were protected by anti-MMP ASO. Method: rats received one month pretreatment with the MMP-9 ASO. A laparotomy was performed, the vessels to the left and median lobe were clamped for one hour, and after 6 hours blood was drawn to measure AST and ALT.

FIG. 3 graphically illustrates data showing that liver-selective MMP-9 inhibition enhances liver regeneration, while systemic MMP inhibition impairs liver regeneration. The data shows that administration of antisense oligonucleotides (ASO) that inhibit liver matrix metalloproteinase-9 (MMP-9) result in a 27% higher liver to body weight ratio as compared to the ASO control (CT ASO). In contrast, systemic inhibition with doxycline, a broad MMP inhibitor impaired liver regeneration by 29% when compared to its control. Methods: Rats were pretreated with ASO for one month or doxycycline for 2 days and then underwent two-thirds hepatectomy. On day 5 the liver and body weights were measured.

FIG. 4 graphically illustrates data showing that liver-selective MMP-9 inhibition accelerates liver regeneration. Normal liver-to-body weight ratio in a simultaneous cohort was 0.42. In the ASO control group, the liver-to-body weight ratio reaches 0.42 on day 7 after partial hepatectomy, whereas in the group pre-treated with MMP-9 ASO, the liver-to-body weight ratio is back to 0.42 on day 4. Methods: rats were pre-treated with ASO for one month, followed by a partial hepatectomy. Cohorts were sacrificed on days 3 through 7 and liver and body weight were measured.

FIG. 5 illustrates a staining for liver sinusoidal endothelial cells (LSECs) with CD31: and that on day 2 after 90% (extended) hepatectomy (middle panel) that there are few sinusoids lined by endothelial cells compared to the control (left panel). In contrast, in the rat pre-treated with the MMP-9 ASO followed by 90% hepatectomy, the number of endothelial cells in the sinusoids is comparable to the control. Conclusion: MMP-9 ASO markedly enhanced re-endothelialization after 90% hepatectomy.

FIG. 6A-B graphically illustrates data showing that liver-selective MMP-9 inhibition increases liver weight and reduces ascites on day 2 after extended hepatectomy; on day 2 after 90% hepatectomy liver weight was increased by 41.5% and ascites was reduced by 56.9% by MMP9 ASO. Methods: rats were pre-treated with control or MMP-9 ASO for one month and then underwent 90% (extended) hepatectomy. On day 2 after hepatectomy rats were weighed, FIG. 6B: ascites was removed and measured, and FIG. 6A the liver was removed and weighed.

FIG. 7A-E graphically illustrate data showing that liver-selective MMP-9 inhibition enhances BM sproc mobilization and engraftment in the liver after partial hepatectomy:

FIG. 7A-D graphically illustrate data showing that liver-selective MMP-9 inhibition enhances BM sproc mobilization and engraftment in the liver after partial hepatectomy:

FIG. 7A illustrates that the number of BM sprocs (CD133+45+31+ cells) was increased after partial hepatectomy in both the group that received MMP-9 ASO, with liver-selective MMP inhibition, but was even higher in the group that received doxycycline, which would impair BM sprocs from mobilizing from the bone marrow. Methods: rats were pre-treated with control or MMP-9 ASO for one month or doxycycline for 2 days and then underwent two-thirds hepatectomy. Bone marrow was harvested on day 2, and the number of CD133+45+31+ cells per femur was measured using immunomagnetic separation of CD133+ cells and flow cytometry for the CD45+31+ fraction of that population;

FIG. 7B examines the number of BM sprocs (CD133+45+31+) in the circulation; MMP-9 ASO significantly increased the number of BM sprocs in the circulation, whereas systemic inhibition of MMP by doxycycline significantly reduced the number of BM sprocs that had been mobilized. Methods: rats were pre-treated with control or MMP-9 ASO for one month or doxycycline for 2 days and then underwent two-thirds hepatectomy. Mononuclear cells were isolated from the blood on day 2 after partial hepatectomy and the number of CD133+45+31+ cells were determined by immunomagnetic separation for CD133+ and flow cytometry for CD31+45+ cells.

FIG. 7C is as 7B except that the population of BM sprocs was CD133+31+45+CXCR7+, a more defined population of cells; additional groups were treated with either intraportal MMP inhibitor (liver-selective) or intraperitoneal MMP inhibitor (systemic MMP inhibition). Methods: rats were pre-treated with control or MMP-9 ASO for one month; for the MMP inhibitor groups, biphenylsulfonyl-D-phenylalanine (Abcam), an MMP-2/9 inhibitor, was infused by an Alzet osmotic pump at 100 μg/hour/kg into either the inferior mesenteric vein, which drains into the portal vein, or into the peritoneal cavity for 2 days prior to partial hepatectomy. Rats then underwent two-thirds hepatectomy. Mononuclear cells were isolated from the blood 6 hour after partial hepatectomy and the number of CD133+45+31+CXCR7+ cells were determined by immunomagnetic separation for CD133+ and flow cytometry for CD31+45+CXCR7+ cells;

FIG. 7D illustrates the percentage of liver sinusoidal endothelial cells (LSECs) that are bone marrow-derived, i.e. derived from BM sprocs (y-axis labeled % GFP+ LSECs). Hepatic engraftment determined as the percentage of bone marrow-derived LSECs after PH is enhanced by MMP-9 ASO or intraportal MMP inhibitor, whereas systemic inhibition of MMP by doxycycline or intraperitoneal MMP inhibitor reduces hepatic engraftment of BM sprocs. Rats were transplanted with bone marrow from a transgenic EGFP rat to allow tracking of bone marrow cells. Rats were treated with one month of ASO, 2 days of doxycycline, or infusion with the MMP inhibitor followed by partial hepatectomy. On day 2 LSECs were isolated and the percentage of GFP+ LSECs was determined by flow cytometry.

FIG. 8 graphically illustrates the percent LSECs as a function of engraftments of infused allogenic sprocs, and examines the concept that impairment of BM release of sprocs will favor engraftment of exogenously infused progenitor cells. The figure graphically illustrates data showing that systemic MMP inhibition significantly increases engraftment of infused, allogeneic sprocs in the liver by day 2. When the percentage of cells derived from the GFP+ allogeneic progenitors was examined at 3 months, 1.5% were GFP+ in the control group versus 14% in the doxycycline group (data not shown). Method: rats were pre-treated with 2 days of doxycycline, 15 mg/kg intra-gastrically twice daily, followed by partial hepatectomy. 1 million sprocs were isolated from the livers of GFP+ rats and infused by tail vein injection. On day 2 and after 3 months LSECs were isolated and the number of GFP+ cells was determined by flow cytometry. Increased ischemia-reperfusion injury in Non-Alcoholic Fatty Liver Disease (NAFLD) due to impaired sdf-1 signaling to bone marrow sprocs can be attenuated by liver-selective MMP inhibition:

FIG. 9 graphically illustrates alanine aminotransferase (ALT) and aspartate aminotransferase (AST) levels in a high-fat-high-fructose (HFHF) diet model of 5, 10 or 15 weeks. This demonstrates the worsening liver disease as the rats remain on the diet longer. Methods: rats were fed a high fat-high fructose diet for 5, 10 or 15 weeks. Blood was drawn and AST and ALT were measured.

FIG. 10 graphically illustrates data confirming that livers with fatty liver have increased susceptibility to ischemia-reperfusion (I/R) and that FR injury is greater with increased fatty liver disease; the graph illustrates that liver enzymes, alanine aminotransferase (ALT) and aspartate aminotransferase (AST), are increased as rats have worsening liver disease, induced by giving 5 10 or 15 weeks of a high fat-high-fructose (HFHF) diet model. Methods: rats were fed a high fat-high fructose diet for 5, 10 or 15 weeks. Rats underwent laparotomy and the vessels to the left and median lobes were clamped for one hour. Six hours later blood was drawn and AST and ALT were measured.

FIG. 11 graphically illustrates the effect of FR injury on the number of BM sprocs per femur in control rats that underwent laparotomy but without FR injury (control/sham); rats given the HFHF diet for 5-15 weeks that underwent laparotomy but without FR injury (HFHF sham); control rats that underwent 1 hour of FR injury (control I/R); and,); rats given the HFHF diet for 5-15 weeks and then underwent 1 hour of FR injury (HFHF FR rats). The figure demonstrates that FR injury increases the number of sprocs in the bone marrow in control rats compared to the sham control, but does not increase the number of sprocs in the bone marrow in HFHF rats compared to the HFHF sham control. Methods: rats were fed a high fat, high fructose diet for 5, 10 or 15 weeks. Rats underwent laparotomy and the vessels to the left and median lobes of the liver were clamped for 1 hours. After 6 hours bone marrow was harvested and the number of CD133+45+31+ cells per femur was determined by immunomagnetic separation followed by flow cytometry.

FIG. 12 graphically illustrates the effect of FR injury on the percentage of proliferating (PCNA+) BM sprocs in control rats that underwent laparotomy but without FR injury (control/sham); rats given the HFHF diet for 5-15 weeks that underwent laparotomy but without I/R injury (HFHF sham); control rats that underwent 1 hour of FR injury (control I/R); and,); rats given the HFHF diet for 5-15 weeks and then underwent 1 hour of FR injury (HFHF FR rats). The figure demonstrates that FR injury increases the percentage of proliferating sprocs in the bone marrow in control rats compared to the sham control, but does not increase the number of proliferating sprocs in the bone marrow in HFHF rats compared to the HFHF sham control. Methods: rats were fed a high fat, high fructose diet for 5, 10 or 15 weeks. Rats underwent laparotomy and the vessels to the left and median lobes of the liver were clamped for 1 hours. After 6 hours bone marrow was harvested and the CD133+45+31+ cells were isolated; the percentage of cells positive for PCNA was determined by flow cytometry.

FIG. 13 graphically illustrates the effect of FR injury on the mobilization of BM sprocs to the circulation after FR Injury in control/sham; HFHF sham; control FR; and, HFHF FR animals; this figure demonstrates that FR injury markedly increases the number of BM sprocs/ml blood in control rats compared to sham, but that there is no increase in the number of circulating BM sprocs in the HFHF diet rats compared to HFHF sham rats. Methods: rats were fed a high fat, high fructose diet for 5, 10 or 15 weeks. Rats underwent laparotomy and the vessels to the left and median lobes of the liver were clamped for 1 hours. After 6 hours blood was drawn, mononuclear cells were separated and the number of CD133+45+31+ cells per ml blood was determined by immunomagnetic separation followed by flow cytometry.

FIG. 14 graphically illustrates that the HFHF diet injures LSECs leading to engraftment of BM sprocs and subsequently to BM-derived LSECs. Method: Rats underwent bone marrow transplantation with bone marrow from transgenic EGFP rats. Rats were then fed a HFHF diet for 5, 10 or 15 weeks. LSECs were isolated and the percentage GFP+ LSECs was determined by flow cytometry.

FIG. 15 graphically illustrates the change in the percentage engraftment of BM-derived LSECs after I/R injury compared to the percentage engraftment with the appropriate control without FR injury. The figure demonstrates that the percentage of BM-derived LSECs increases in control rats that have undergone FR injury, but that there is no increase in rats that have received the HFHF diet Method: Rats underwent bone marrow transplantation with bone marrow from transgenic EGFP rats. Rats were then fed a HFHF diet for 5, 10 or 15 weeks. Rats underwent laparotomy, the vessels to the left and median lobes of the liver were clamped for one hour, and at 6 hours LSECs were isolated and the percentage GFP+ LSECs was determined by flow cytometry.

FIG. 16 graphically illustrates data showing that allogeneic sprocs cannot be used to rescue in HFHF model. This graph shows that allogeneic GFP+ sprocs infused after 1 hour of FR injury engraft in control rats, but that there is little engraftment in rats that received 5 weeks of the HFHF diet. Methods: Rats were fed a HFHF diet for 5 weeks. Rats underwent laparotomy and the vessels to the left and median lobes of the liver were clamped for one hour. One million sprocs isolated from the liver of transgenic GFP rats were infused into the tail vein. On day 2 LSECs were isolated and the percentage GFP+ LSECs was determined by flow cytometry

FIG. 17A-B graphically illustrate data showing that liver-selective MMP-9 inhibition attenuates FR injury in control rats and in 5 and 10 week HFHF model rats, but not in the 15 week model; the liver panel in FIG. 17A demonstrates levels of ALT, and FIG. 17B demonstrates AST levels, both are liver enzymes that reflect liver injury. Method: Rats received 5, 10 or 15 weeks of a HFHF diet and during the last month of this, rats received MMP-9 ASO or an ASO control. Rats underwent laparotomy, the vessels to the left and median lobes of the liver were clamped for one hour, and at 6 hours blood was drawn for AST and ALT measurement.

FIG. 18A-D (or FIG. 1 of Example 1) graphically illustrate hepatic and bone marrow MMP-9 after partial hepatectomy (PH): FIG. 18(A) graphically illustrates images of immunoblots of hepatic pro-MMP-9 protein expression assessed 6 hours after PH;

FIG. 18B graphically illustrates a quantification of FIG. 18A, where the data shows increased hepatic MMP-9 protein expression after PH and this increase is abrogated by MMP-9 ASO, doxycycline, and intraportal or intraperitioneal administration of an MMP-2/9 inhibitor (n=3); FIG. 18C graphically illustrates images of immunoblots of pro-MMP-9 and MMP-9 in the BM extracellular fluid; FIG. 18D graphically illustrates quantification of activation of BM MMP-9, expressed as the ratio of MMP-9/pro-MMP-9 in BM extracellular fluid, increases after PH (n=3); there is no significant difference in the increase after PH with pretreatment by control ASO vs MMP-9 ASO.

FIG. 19A-F (or FIG. 2 of Example 1) graphically illustrate liver-selective MMP-9 inhibition accelerates liver regeneration after two-thirds partial hepatectomy (PH). FIG. 19A graphically illustrates a time-course from day 3-day 7: pretreatment with MMP-9 ASO accelerates liver regeneration after PH compared to scrambled control ASO (n=3). The dotted line is the average liver-to-body weight ratio of untreated control littermates. FIG. 19B graphically illustrates the percentage of proliferating hepatocytes and non-parenchymal cells in the sinusoids on day 2 (ki-67) (n=3). FIG. 19C graphically illustrates The number of hepatocytes proliferating in each zone from day 2-day 6 is shown for control ASO and MMP-9 ASO pretreated prior to PH (n=3 for each day).

FIG. 19D graphically illustrates the number of non-parenchymal cells proliferating in each zone from day 2-day 6 is shown for control ASO and MMP-9 ASO pretreated prior to PH (n=3 for each day). FIG. 19E graphically illustrates hepatocyte proliferation (ki-67) on day 2 after PH is enhanced by liver-selective MMP inhibition with MMP-9 ASO or by intraportal MMPi compared to intraperitoneal MMPi, whereas systemic inhibition with doxycycline reduces hepatocyte proliferation (n=3). FIG. 19F graphically illustrates MMP-9 ASO increases liver/body weight ratio by 27% on day 5 after PH, whereas doxycycline reduces liver/body weight ratio by 29% (n=3). Abbreviations: CT, control; Dox, doxycycline; MMPi 2/9, inhibitor of MMP 2 and 9; PH, partial hepatectomy; PP, periportal, ML, midlobular, CL, centrilobular; / indicates two treatments, e.g. MMP ASO/PH is MMP ASO plus partial hepatectomy. Analysis by ANOVA was statistically significant and analyzed post-hoc by Fisher's least significant difference. Levels of statistical significance are * p<0.05, ** p<0.01, *** p<0.001, and **** p<0.0001. Unless otherwise indicated, significance is based on comparison with the appropriate control. Unprocessed original scans of blots are shown in FIG. 28A-B (Supporting Figure S2B) and FIG. 29-B (Supporting FIG. 2C).

FIG. 20A-D (or FIG. 3 of Example 1) illustrates liver-selective MMP-9 inhibition prevents ischemia-reperfusion (FR) injury. FIG. 20A graphically illustrates ALT (n=3) and FIG. 20A graphically illustrates AST in littermate controls, and after pretreatment with control ASO or MMP-9 ASO followed by FR injury, assessed by colorimetric assay (n=3). FIG. 20C illustrates a high power hematoxylin-eosin (H & E) stain of pericentral lobule: control ASO/FR injury (left panel) shows early hepatocyte injury with clearing of cytoplasm, lobular disarray, and lack of LSECs compared to normal appearing liver without hepatocyte injury or loss of LSECs (arrowheads) in the MMP-ASO/I/R group (right panel). FIG. 20D illustrates a low power H & E stain demonstrates widespread hepatocyte changes with sparing around portal tract (vessel bottom left quadrant) and of terminal hepatocytes near the central vein in control ASO/FR injury (left panel) versus no visible injury in MMP-9 ASO/FR injury (right panel). Analysis by ANOVA was statistically significant and analyzed post-hoc by Fisher's least significant difference. **** p<0.0001 compared to MMP-9 ASO/FR.

FIG. 21A-C (or FIG. 4 of Example 1) illustrate liver-selective MMP-9 inhibition restores endothelial integrity, accelerates liver regeneration, and reduces ascites on day 2 after extended (90%) hepatectomy. FIG. 21A illustrates a CD31 staining of normal liver (left panel), and on day 2 after extended hepatectomy in control ASO pretreated (middle panel) or MMP-9 ASO pretreated rats (right panel); images are centered on the portal tract. FIG. 21B graphically illustrates hepatocyte proliferation and FIG. 21C graphically illustrates liver weight and ascites (n=3). *p<0.05 compared to control ASO by unpaired t-test.

FIG. 22A-D (or FIG. 5 of Example 1) graphically illustrate recruitment of BM sprocs after PH is enhanced by liver-selective MMP inhibition and reduced by systemic MMP inhibition. FIG. 22A graphically illustrates MMP-9-ASO (liver selective) and doxycycline (systemic) inhibition of MMP increase the number of sprocs in the BM (n=3). FIG. 22B graphically illustrates MMP-9 ASO increases and systemic doxycycline decreases BM sproc mobilization to the circulation (n=3). FIG. 22C graphically illustrates The number of circulating CXCR7+ sprocs are increased by liver-selective MMP inhibition (MMP-9 ASO or intraportal infusion of an MMP2/9 inhibitor), but reduced by systemic MMP inhibition (doxycycline or intraperitoneal MMP2/9 inhibitor) (n=3). FIG. 22D graphically illustrates hepatic engraftment after PH is enhanced by liver-selective MMP inhibition (MMP-9 ASO or intraportal MMP2/9 inhibitor), but reduced by systemic MMP inhibition (doxycycline or intraperitoneal MMP2/9 inhibitor) (n=3). FIG. 22A-C are 6 hour time points; engraftment in FIG. 22D was determined at 24 hours. Analysis by ANOVA was statistically significant and analyzed post-hoc by Fisher's least significant difference. Levels of statistical significance are * p<0.05, ** p<0.01, *** p<0.001, and **** p<0.0001. Unless otherwise indicated, significance is based on comparison with the appropriate control.

FIG. 23A-D (or FIG. 6 of Example 1) illustrates that MMP-9 cleaves VEGF₁₆₄ with attenuation of sdf-1 expression. Increased MMP-9 after PH causes proteolytic cleavage of VEGF₁₆₄, resulting in formation of a 14 kDa fragment, assessed 6 hours after PH. Pretreatment with MMP-9 ASO prevents the formation of the 14 kDa product and thereby increases expression of VEGF₁₆₄ and the downstream signaling partner of VEGF, sdf-1. FIG. 23A illustrates an immunoblot of VEGF₁₆₄, VEGF₁₆₄ cleavage fragment, and sdf-1 (n=3). FIG. 23B illustrates quantitation of the immunoblots showing the 17 kDa cleavage product of VEGF₁₆₄, with an increase after PH that is blocked by MMP-9 ASO pretreatment; FIG. 23C-D illustrate that VEGF₁₆₄ and sdf-1, with an increase after PH that is further increased after MMP-9 ASO pretreatment. Analysis by ANOVA was statistically significant and analyzed post-hoc by Fisher's least significant difference. Levels of statistical significance are * p<0.05, ** p<0.01, *** p<0.001, and **** p<0.0001. Unprocessed original scans of blots are shown in FIG. 34A-D (Supporting Figure S7).

FIG. 24 (or FIG. 7 of Example 1) graphically illustrates a model of stem cell therapy, where systemic MMP inhibition with doxycycline (Dox) followed by PH and infusion of GFP+ allogeneic sprocs. Doxycycline enhances engraftment of infused sprocs, expressed as the percentage of GFP+ LSECs two days and three months later (n=3). **** p<0.0001 and * P<0.05 compared to control by unpaired t-test.

FIG. 25A-B (or FIG. 8 of Example 1) schematically illustrates Molecular pathway diagram contrasting the effect of liver-selective and systemic MMP-9 inhibition. FIG. 25A illustrates liver-selective MMP-9 inhibition prevents hepatic MMP-9 from proteolytically digesting hepatic VEGF after liver injury or partial hepatectomy. This permits the hepatic VEGF-sdf1 pathway to recruit CXCR7+ sprocs from the bone marrow, but does not inhibit MMP-9 activity in the bone marrow needed to mobilize sprocs. FIG. 25B illustrates systemic inhibition of MMP prevents proteolytic cleavage of VEGF by hepatic MMP-9, thus preserving the VEGF-sdf1 pathway, but prevents sprocs from leaving the bone marrow by inhibiting the bone marrow MMP activity needed for release of the sprocs. The net effect is to reduce circulating sprocs and impair recruitment of sprocs to the liver.

FIG. 26A-R (or supporting Figure S1) graphically illustrate representative images of Flow Cytometry assay of bone marrow and circulating sprocs;

FIG. 26A-I illustrate sorting of CD133+CD31+CD45+ bone marrow cells;

FIG. 26J-R illustrate sorting of CD133+CD31+CD45+ blood cells; bone marrow or circulating mononuclear cells underwent positive selection with CD133 immunomagnetic beads using AUTOMACS PRO™ (AutoMACS Pro™), and then stained for CD31 (PE) and CD45 (FITC);

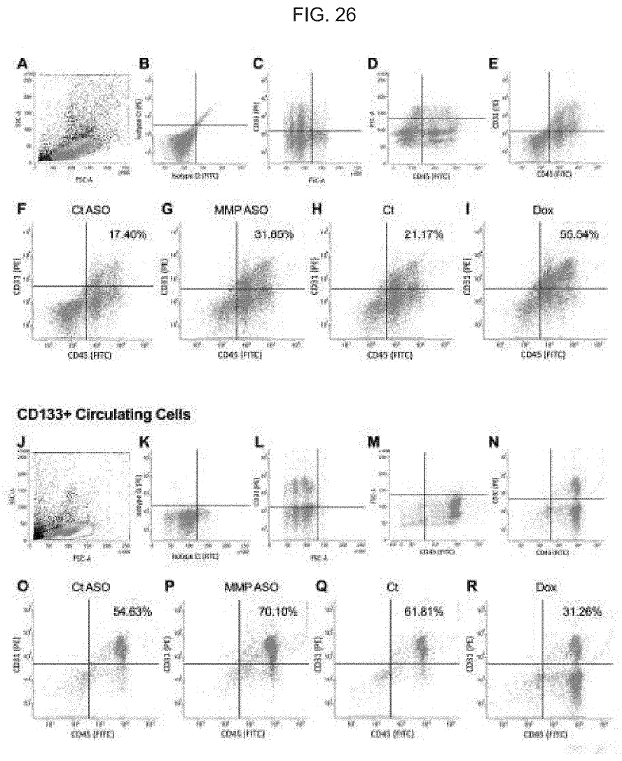

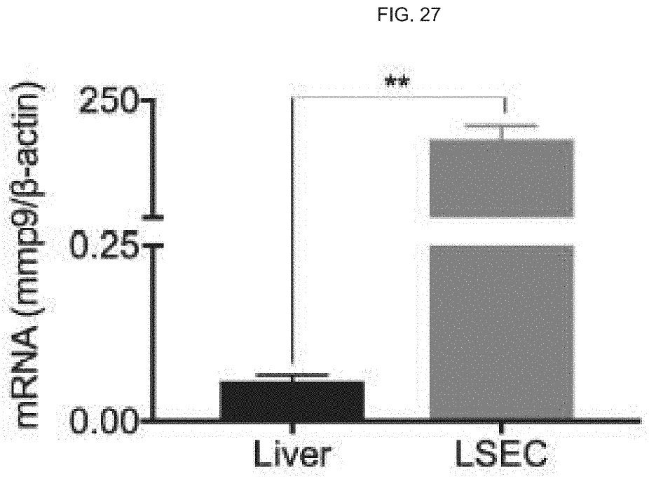

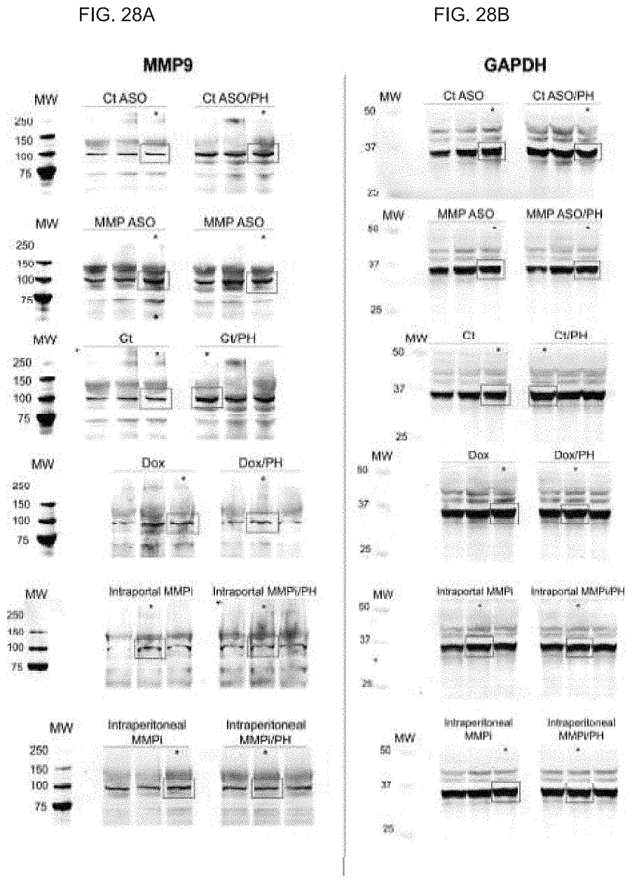

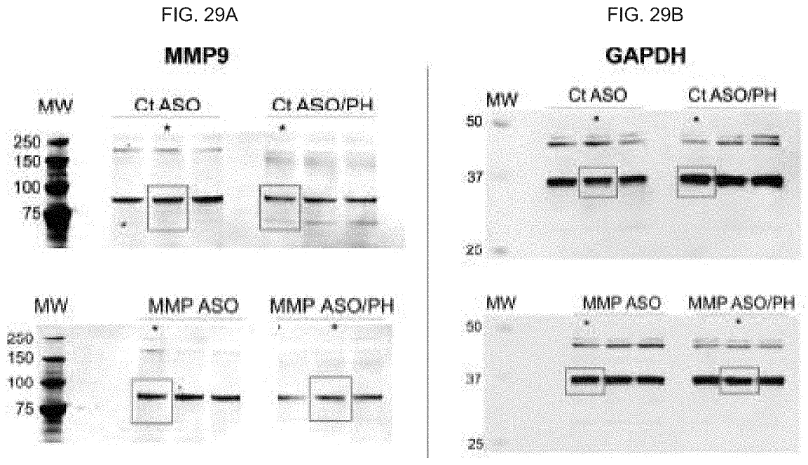

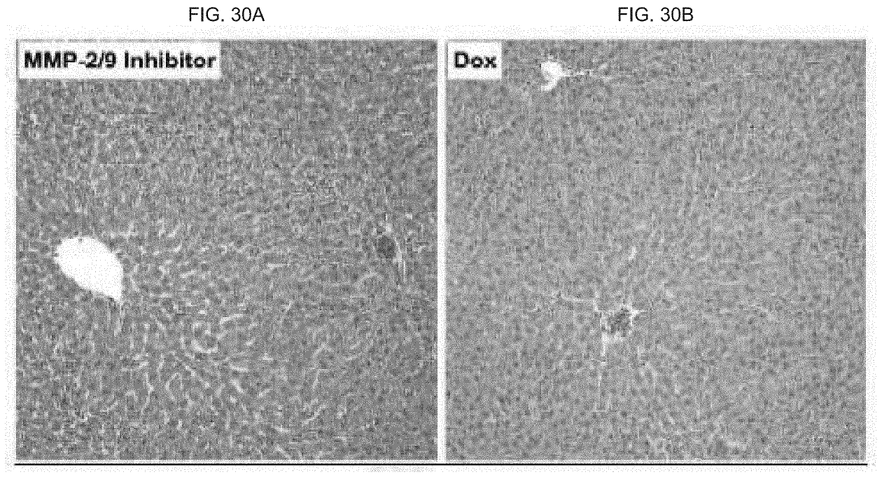

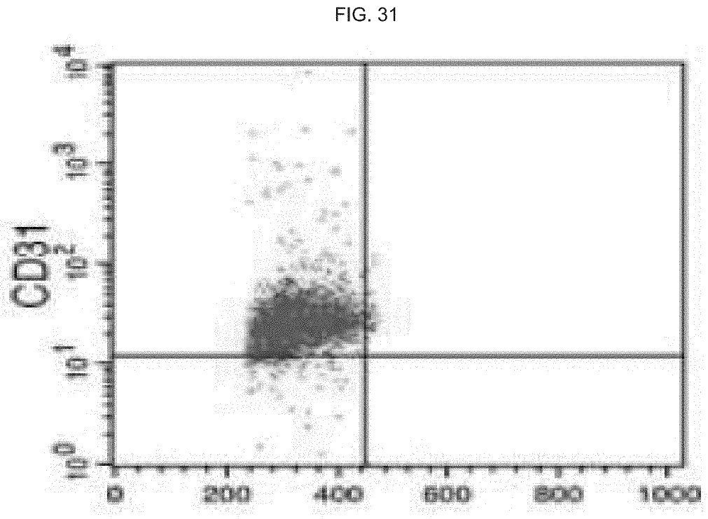

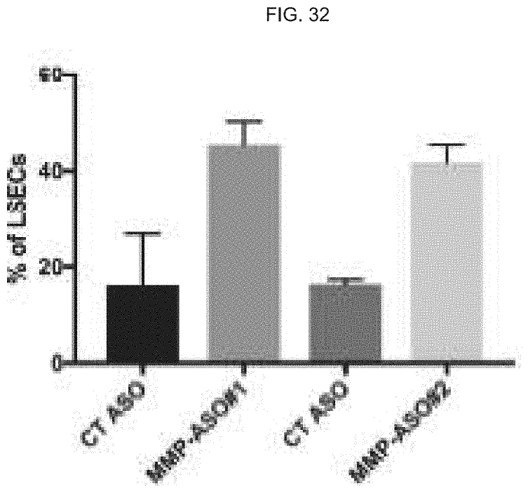

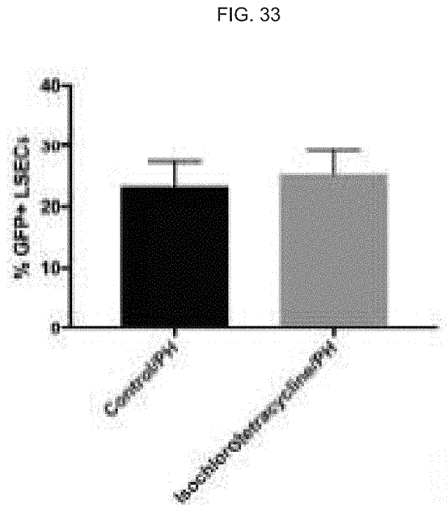

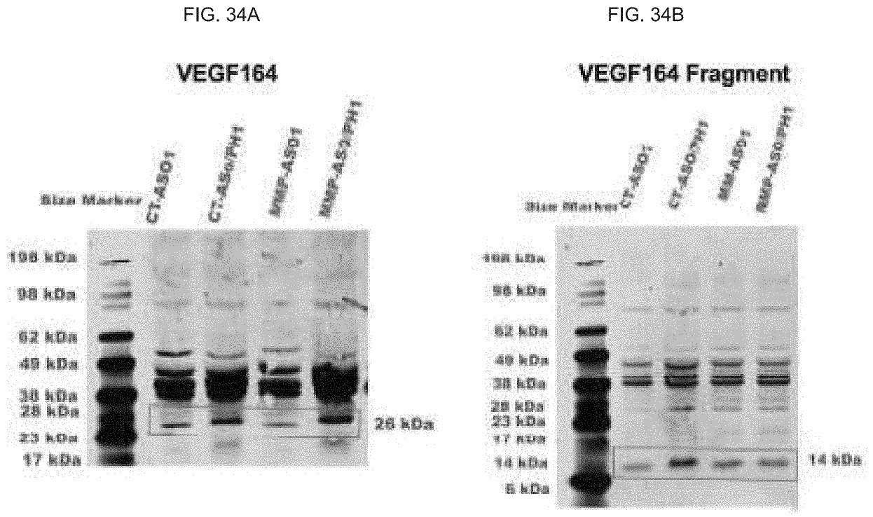

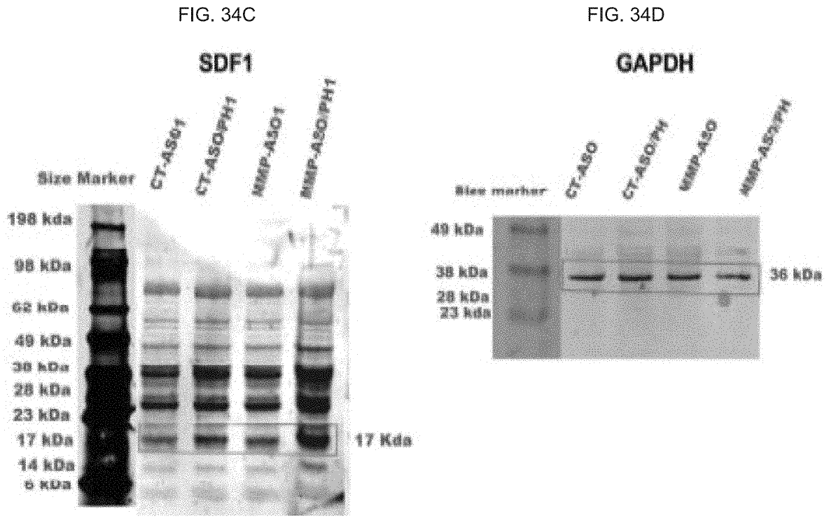

Cell suspensions in FIG. 26A-R were sorted using the following gating strategy: (A/J) Target population was gated based on size x granularity and used for further analysis. (B/K) Quadrants were designed based on the isotype control staining (PE and FITC), which differentiated non-specific background signal from specific antibody signal. The presence of (C/L) CD31+ and (D/M) CD45+ populations was confirmed, and then the double positive cells were analyzed and quantified (E/N). This analysis was performed in using animals treated with Ct ASO (F/O), MMP ASO (G/P), Ct (H/Q) and Doxycycline (FR).

FIG. 27 (or supporting Figure S2A) graphically illustrates data showing that LSEC is a major source of MMP-9 in the liver. MMP-9 gene expression was measured using RNA extracted from normal whole liver and from normal LSECs. LSECs express significantly higher levels of MMP-9 compared to whole liver, suggesting that LSECs a major producer of MMP-9 in the liver. These results are consistent with our earlier findings, where we compared the gelatinolytic activity of LSECs, hepatocytes, Kupffer cells and HSCs in vitro, and showed that LSECs are the only significant source of MMP activity among these liver cells (1).

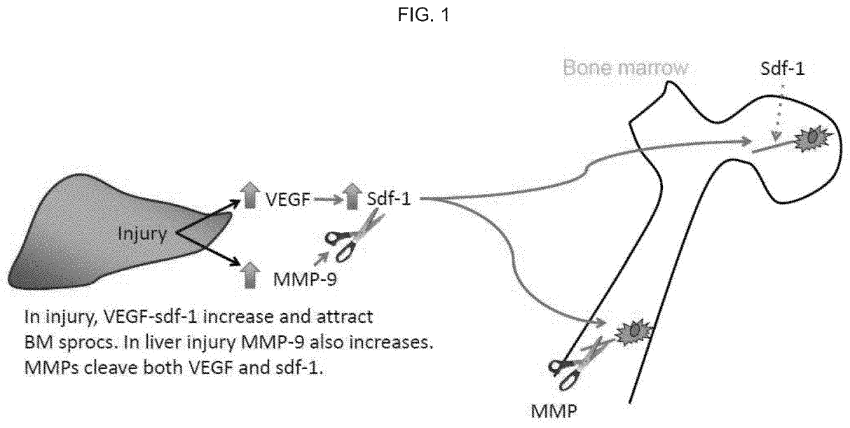

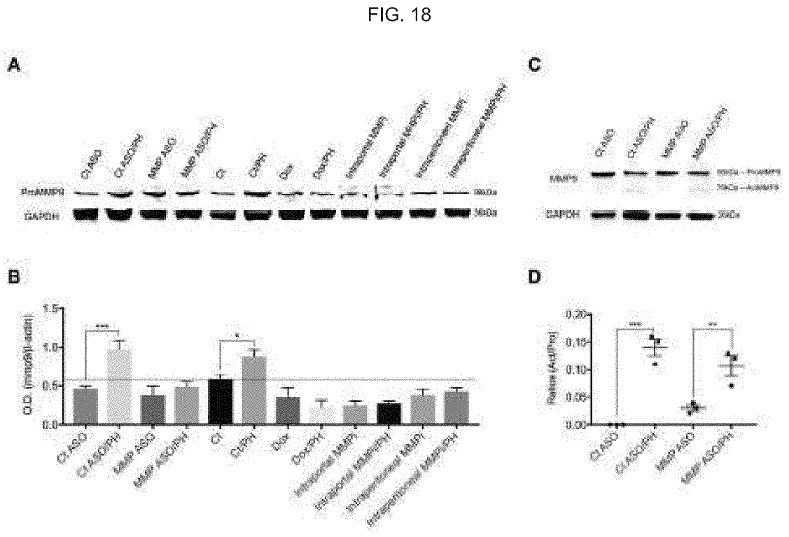

FIG. 28A-B (or supporting Figure S2B) illustrate images of unprocessed original scans of immunoblots related to FIG. 18A (or FIG. 1A of Example 1), FIG. 28A showing MMP9, and FIG. 28B showing GAPDH. Uncropped images of all Western blots. Red rectangles indicate portion of image used on indicated figure. Molecular size markers in kDa.

FIG. 29A-B (or supporting Figure S2C) illustrate unprocessed original scans of Immunoblots related to FIG. 18B (or FIG. 1B of Example 1), FIG. 29A showing MMP9, and FIG. 29B showing GAPDH. Uncropped images of all Western blots. Red rectangles indicate portion of image used on indicated figure. Molecular size markers in kDa.

FIG. 30A-B (or supporting Figure S3A): FIG. 30A-B illustrate images of the cytotoxicity of MMP inhibitors; histology showed no evidence of toxicity from the MMP-2/9 inhibitor (FIG. 30A) or coxycycline (FIG. 30B).

FIG. 31 (or supporting Figure S3B) graphically illustrates data showing the CD31⁺ fraction of CD133⁺ liver cells. Whole liver was digested and CD133⁺ cells were isolated by immunomagnetic selection. Cells were stained for CD31. At least 94% of the CD133⁺ cells were CD31⁺.

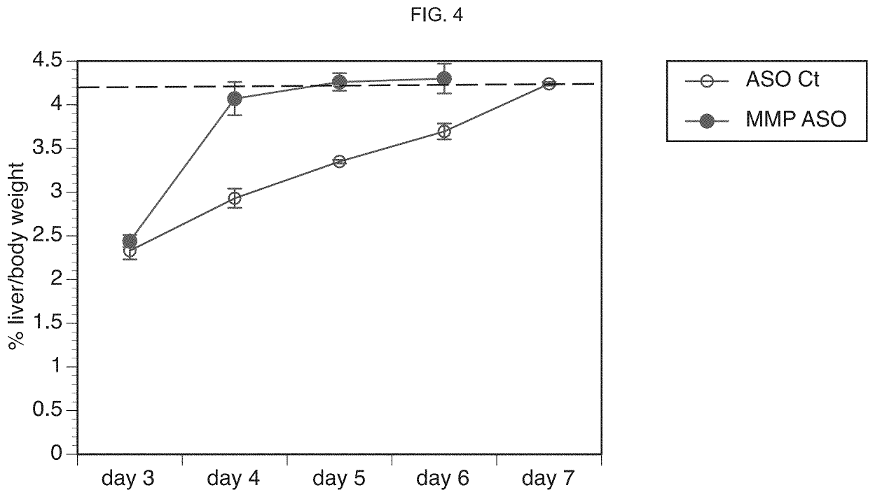

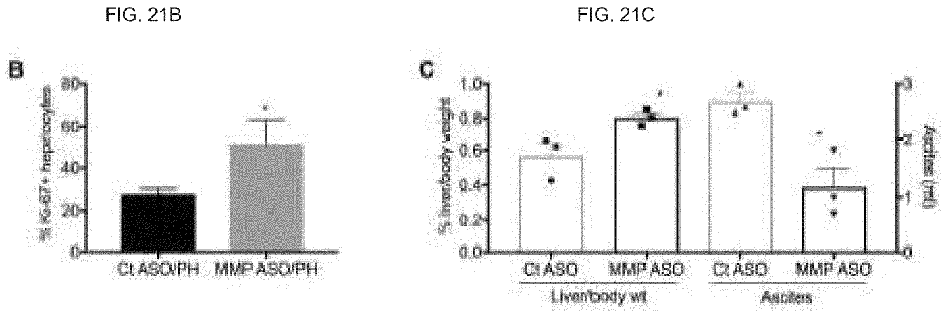

FIG. 32 (or supporting Figure S4) graphically illustrates data showing confirmatory MMP-9 ASO. A second MMP-9 ASO (Ions Pharmaceuticals) confirmed that the effect of pretreatment with the MMP-9 ASO was through MMP-9 inhibition. There was no significant difference in engraftment of BM sprocs after PH between the effect of MMP-9 ASO #1 (also shown in FIG. 21D (FIG. 4D of Example 1)) and a second MMP-9 ASO labeled MMP-9 ASO #2.

FIG. 33 (or supporting Figure S6) graphically illustrates data showing the effect of isochlorotetracycline pretreatment on BM sproc engraftment after partial hepatectomy. Isochlorotetracycline shares an antibiotic effect with doxycycline, but has only weak MMP inhibitory activity. Thus this is a control for the antibiotic effect of doxycycline on recruitment and engraftment.

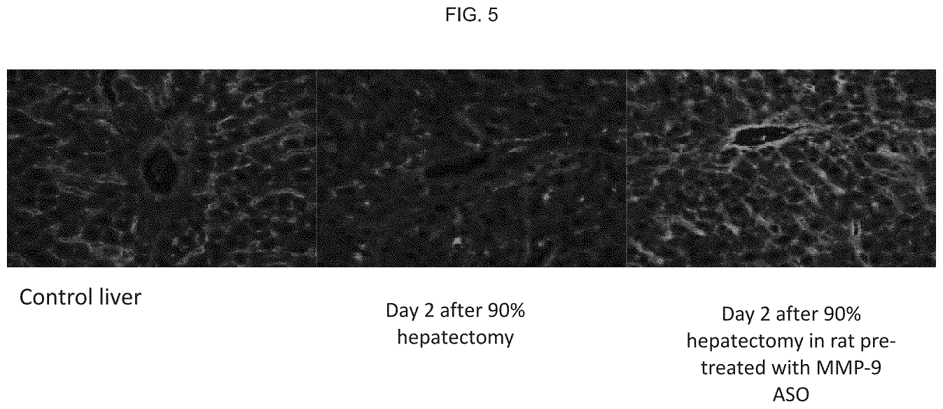

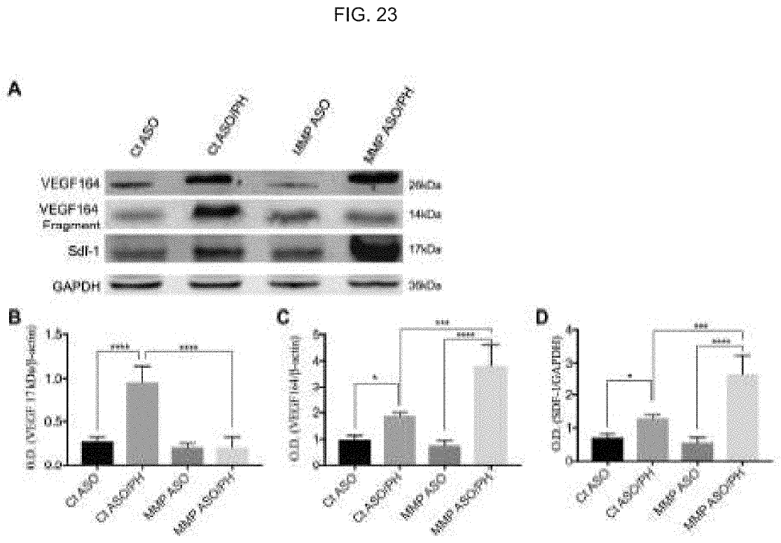

FIG. 34A-D (or supporting Figure S7) illustrates unprocessed original scans of immunoblots related to FIG. 23A-D (or FIG. 5 of Example 1), where the (red) rectangles indicate the portion of the image used for FIG. 23A-D; molecular size markers in kDa; where FIG. 34A shows VEGF 164; FIG. 34B shows VEGF 164 fragment; FIG. 34C shows SDF1; and FIG. 34D shows GAPDH.

Like reference symbols in the various drawings indicate like elements.

### DETAILED DESCRIPTION

In alternative embodiments, provided are compositions, including pharmaceutical compositions and formulations, products of manufacture and kits, and methods, for: enhancing or accelerating liver regeneration, optionally enhancing or accelerating liver regeneration after tissue injury or liver resection; enhancing or accelerating tissue or organ repair, e.g., a liver, brain, lung, pancreas, kidney, skin or heart repair, optionally enhancing or accelerating tissue or organ repair after a trauma, an injury or an infection, wherein optionally the injury is an ischemia-reperfusion injury such as e.g., a heart attack or a stroke; or, reducing the extent of or abolishing ischemia-reperfusion injury in a tissue or organ, e.g., in a normal liver or a fatty liver, or a cadaver or donor liver or transplant organ, or optionally a cadaveric or donor lung, heart, pancreas, skin, or kidney, comprising: administering to an individual in need thereof, a compound or composition capable of inhibiting or decreasing the expression or activity of a matrix metalloproteinase (MMP) in a liver-specific or liver-selective manner, or administering to the liver of an individual in need thereof a compound or composition capable of inhibiting or decreasing the expression or activity of a matrix metallo-proteinase (MMP), or inhibiting or decreasing the expression or activity of a matrix metallo-proteinase (MMP) in an end-organ specific manner.

In alternative embodiments, provided are compositions, including pharmaceutical compositions and formulations, products of manufacture and kits, and methods, for: enhancing infused or exogenous progenitor cell, optionally bone marrow endothelial progenitor cell, engraftment in an organ or tissue, wherein optionally the organ or tissue is a heart, lung, pancreas, skin, kidney or a liver, by preventing or inhibiting proteolytic cleavage of a bone marrow sdf1, and thereby reducing release of an endogenous progenitor cell, optionally an endogenous bone marrow endothelial progenitor cell, from the bone marrow, to decrease competition for engraftment of the infused or exogenous progenitor cell by the endogenous progenitor cell, comprising administering to an individual in need thereof a compound or composition capable of inhibiting or decreasing the expression or activity of a matrix metalloproteinase (MMP) in a systemic manner, or in a bone marrow specific manner, or treating the organ or tissue with the compound or composition capable of inhibiting or decreasing the expression or activity of the MMP, and optionally the treatment is in a perfusion bath.

Matrix metalloproteinases (MMPs) proteolytically cleave the VEGF-sdf1 signaling pathway generating a much less potent isoform of VEGF and reducing the chemoattractant activity of sdf-1. Inhibition of hepatic MMP activity after liver injury enhances VEGF-sdf1 recruitment and repair by BM endothelial progenitor cell (sprocs). On the other hand, proteolytic cleavage by MMP-9 of BM sdf1 is necessary for release of cells from the bone marrow. Thus, liver-selective inhibition of MMP activity improves BM endothelial progenitor cell recruitment and engraftment in the liver, whereas systemic MMP inhibition impairs BM endothelial progenitor cell mobilization from the BM and diminishes repair of liver injury.

Our research shows that (1) Liver-selective inhibition of hepatic MMP-9 with sparing of BM MMP-9 reduces liver injury and accelerates liver regeneration, whereas systemic inhibition of MMP is detrimental. (2) Engraftment of infused endothelial progenitor cell is enhanced by systemic MMP inhibition and by reducing release of endothelial progenitor cells from the BM and thereby decreasing competition for engraftment. We have shown the therapeutic benefit of liver-selective MMP-9 inhibition in models of ischemia-reperfusion injury, two-thirds partial hepatectomy, and in 90% hepatectomy as a model of small-for-size syndrome.

Liver selective matrix metalloproteinase (MMP) inhibition abolished ischemia-reperfusion injury in normal liver and markedly decreased ischemia-reperfusion injury in fatty liver. Liver selective MMP inhibition improves liver regeneration from small for size injury and improves liver function. Small for size injury: in living donor transplantation or in split cadaver donor transplantation, a portion of the liver is transplanted. Given that the circulation is meant for a larger organ, the microcirculation of the grafted liver is damaged.

### Cadaver or Donor Liver Treatment

In alternative embodiments, in cadaver donor tissue or organ, e.g., liver, lung, kidney, muscle, bone, skin, trachea, arterial or venous blood vessels, intestine, spinal cord, skin, heart or pancreas, transplantation an MMP inhibitor could either be given to the donor before removing the tissue or organ or could be part of a warm perfusion system or bath of the tissue or organ before the tissue or organ was transplanted into the recipient. In living-related donor transplantation, organ-specific MMP inhibition could be achieved either by administering MMP-9 ASO to the donor or by slowly infusing a pharmacological MMP-9 inhibitor into the tissue or organ circulation at the time the portion of the tissue or organ was removed from the donor.

### Antibodies

In alternative embodiments, provided are compositions and methods for inhibiting or decreasing the expression or activity of an MMP in a tissue or an organ, e.g., a liver MMP, optionally in an organ-specific or an organ-selective manner, by, e.g. administering a small molecule, a peptide or a polypeptide, e.g., an antibody or fragment thereof or equivalent thereof, capable of specifically binding or otherwise inhibiting the activity or expression of a specific organ or tissue MMP, e.g., a liver MMP, and/or is capable of inhibiting or decreasing the activity (in the organ or tissue, e.g., liver) of MMP. In alternative embodiments, the MMP includes e.g., an MMP-1, MMP-2, MMP-3, MMP-9, MMP-11, and/or MMP-13, or any combination thereof.

Antibodies or fragments thereof capable of specifically binding MMP can be designed using Homo sapiens MMP proteins or fragments thereof

### Enhancing Stem Cell Treatments

In alternative embodiments, provided are compositions, including pharmaceutical compositions and formulations, products of manufacture and kits, and methods, for supporting stem cell therapies where an exogenous cell is introduced into (administered to), or an endogenous cell is reintroduced into, an individual in need thereof. Compositions and methods as described herein can be used to support stem cells intended to target and repopulate any tissue or organ, including liver, heart, lung, pancreas, skin, kidney or nerve (e.g., spinal cord), skin or other tissue.

In alternative embodiments, stem cell therapy supporting compositions and methods as provided herein are used to enhance infused or exogenous progenitor cells, optionally bone marrow endothelial progenitor cell, engraftment in an organ or tissue, e.g., a heart, lung, pancreas, kidney, skin, nerve tissue or a liver, by preserving the chemoattractant effect of sdf1 in the organ while also preventing or inhibiting proteolytic cleavage of a bone marrow sdf1, and thereby reducing release of an endogenous progenitor cell, optionally an endogenous bone marrow endothelial progenitor cell, from the bone marrow, to decrease competition for engraftment of the infused or exogenous progenitor cell by the endogenous progenitor cell.

In alternative embodiments, methods and uses as provided herein are used to enhance infused or exogenous progenitor cells (e.g., endothelial progenitor cells) engraftment in an organ or tissue, e.g., in a heart, lung, pancreas, kidney, skin or a liver, by preventing or inhibiting proteolytic cleavage of a sdf1 in that organ or tissue. Data supports end-organ inhibition of MMP as beneficial for many organs. The VEGF-sdf1 pathway (inhibited by MMP) attracts progenitor cells to a variety of organs or tissues with beneficial results in a variety of organs or tissues. This pathway attracts endothelial progenitor cells to the kidney after ischemia-reperfusion injury (reference 1) (all references listed below), to the blood vessels that supply nerves in a model of diabetic peripheral neuropathy (reference 2), may improve regeneration after spinal cord injury (reference 3), may promote bone fracture healing (references 4 and 8), may help restore lung structure in neonates with lung damage from hyperoxia (reference 6), may improve the vascular niche for neural stem cells and thereby improve recovery from cerebral infarction (reference 7), may improve post-injury regeneration of vasculature in hemorrhagic stroke (reference 17) and ischemic stroke (reference 16), may improve the impaired formation of new blood vessels in diabetes mellitus (reference 9), may improve blood vessel formation in ischemic limbs (reference 10 and 18) and skin flaps (reference 11), may improve blood vessel formation in coronary artery disease and refractory angina (reference 12, 14 and reviewed in reference 13), may promote lung repair in acute respiratory distress syndrome (reference 15).

The studies cited above demonstrate a role for the VEGF-sdf1 pathway in recruiting endothelial progenitor cells in many organs. In addition to the liver, MMPs rise after injury in several organs, including kidney (reference 18 and 19), lung (references 20 and 21), pancreas (reference 22), heart (references 23, 24, and 25), vascular wall (references 26 and 27), and brain (reference 19 and 28). Taken with data described herein that demonstrate that end-organ MMP activity reduces recruitment of bone marrow endothelial progenitor cells, this supports the concept that end-organ inhibition of MMP will be beneficial for many organs. MMP is needed for mobilization of bone marrow cells (References 29 and 30); this supports the concept that MMP inhibition needs to spare bone marrow MMP to allow mobilization of bone marrow endothelial progenitor cells but that inhibiting bone marrow MMP will prevent competition when the goal is to achieve engraftment of infused progenitor cells. These studies therefore support methods provided herein that comprise the selective inhibition of MMP with sparing of bone marrow MMP is protective in injuries and disease of the lungs, spinal cord, kidneys, pancreas, heart, brain, as well as peripheral vascular disease and diabetic neuropathy.

### Antisense Inhibitory Nucleic Acid Molecules

In alternative embodiments, MMP-inhibiting pharmaceutical compositions and formulations methods as provided herein are administered to an individual in need thereof in an amount sufficient to practice methods as provided herein, e.g., for stimulating liver regeneration in an individual, or for enhancing or accelerating organ or tissue regeneration, optionally enhancing or accelerating organ or tissue, e.g., liver, regeneration after tissue injury or liver resection. In alternative embodiments, MMP-inhibiting pharmaceutical compositions and formulations methods as provided herein are administered to an individual in need thereof in an amount sufficient to ameliorate injury to an organ or tissue, e.g., to a liver, brain, nerve tissue, pancreas, lung, kidney, skin or heart.

In alternative embodiments, provided are compositions and methods for, e.g., enhancing or accelerating liver regeneration, optionally enhancing or accelerating liver regeneration after tissue injury or liver resection, in an individual, by targeting and inhibiting the expression or activity of an MMP, optionally in a liver-specific or liver-selective manner, e.g., targeting and inhibiting the expression or activity of Homo sapiens MMP, by, e.g., administering MMP-inhibiting nucleic acids, e.g., an antisense morpholino oligonucleotide (MO), an miRNA, an siRNA and the like.

In alternative embodiments, compositions and methods as provided herein comprise use of an inhibitory nucleic acid molecule or an antisense oligonucleotide inhibitory to expression of an MMP, including e.g., an MMP-1, MMP-2, MMP-3, MMP-9, MMP-11, and/or MMP-13, or any combination thereof. In alternative embodiments, compositions and methods as provided herein comprise use of an inhibitory nucleic acid molecule or anti sense oligonucleotide inhibitory to expression of an MMP, comprising: an RNAi inhibitory nucleic acid molecule, a double-stranded RNA (dsRNA) molecule, a small interfering RNA (siRNA), a microRNA (miRNA) and/or a short hairpin RNA (shRNA), or a ribozyme.

Naturally occurring or synthetic nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol. Appl. Pharmacol. 144:189-197; Antisense Therapeutics, ed. Agrawal (Humana Press, Totowa, N.J., 1996). Antisense oligonucleotides having synthetic DNA backbone analogues can also include phosphoro-dithioate, methylphosphonate, phosphoramidate, alkyl phosphotriester, sulfamate, 3′-thioacetal, methylene (methylimino), 3′-N-carbamate, and morpholino carbamate nucleic acids.

RNA Interference (RNAi)

In alternative embodiments, provided are RNAi inhibitory nucleic acid molecules capable of decreasing or inhibiting expression of one or a set of MMP transcripts or proteins, including e.g., decreasing or inhibiting expression of MMP-1, MMP-2, MMP-3, MMP-9, MMP-11, and/or MMP-13, or any combination thereof, optionally in a liver-specific or liver-selective manner, and including e.g., decreasing or inhibiting expression of the transcript (mRNA, message) or isoform or isoforms thereof. In one aspect, the RNAi molecule comprises a double-stranded RNA (dsRNA) molecule. The RNAi molecule can comprise a double-stranded RNA (dsRNA) molecule, e.g., siRNA, miRNA (microRNA) and/or short hairpin RNA (shRNA) molecules.

In alternative aspects, the RNAi is about 11, 12, 13, 14, 15, 16, 17, 18, 19, 20, 21, 22, 23, 24, 25 or more duplex nucleotides in length. While the methods provided herein are not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi, e.g., siRNA for inhibiting transcription and/or miRNA to inhibit translation, is the breaking of a double-stranded RNA (dsRNA) matching a specific gene sequence into short pieces called short interfering RNA, which trigger the degradation of mRNA that matches its sequence.

In one aspect, intracellular introduction of the RNAi (e.g., miRNA or siRNA) is by internalization of a target cell specific ligand bonded to an RNA binding protein comprising an RNAi (e.g., microRNA) is adsorbed. The ligand can be specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, e.g., U.S. Patent App. Pub. Nos. 20060030003; 20060025361; 20060019286; 20060019258. In one aspect, provided are lipid-based formulations for delivering, e.g., introducing nucleic acids used in methods as provided herein, as nucleic acid-lipid particles comprising an RNAi molecule to a cell, see, e.g., U.S. Patent App. Pub. No. 20060008910.

Methods for making and using RNAi molecules, e.g., siRNA and/or miRNA, for selectively degrade RNA are well known in the art, see, e.g., U.S. Pat. Nos. 6,506,559; 6,511,824; 6,515,109; 6,489,127.

Methods for making expression constructs, e.g., vectors or plasmids, from which an inhibitory polynucleotide (e.g., a duplex siRNA) is transcribed are well known and routine. A regulatory region (e.g., promoter, enhancer, silencer, splice donor, acceptor, etc.) can be used to transcribe an RNA strand or RNA strands of an inhibitory polynucleotide from an expression construct. When making a duplex siRNA inhibitory molecule, the sense and antisense strands of the targeted portion of the targeted IRES can be transcribed as two separate RNA strands that will anneal together, or as a single RNA strand that will form a hairpin loop and anneal with itself. For example, a construct targeting a portion of a gene, e.g., an MMP coding sequence or transcriptional activation sequence, is inserted between two promoters (e.g., mammalian, viral, human, tissue specific, constitutive or other type of promoter) such that transcription occurs bidirectionally and will result in complementary RNA strands that may subsequently anneal to form an inhibitory siRNA used to practice methods as provided herein.

Alternatively, a targeted portion of a gene, coding sequence, promoter or transcript can be designed as a first and second antisense binding region together on a single expression vector; for example, comprising a first coding region of a targeted gene in sense orientation relative to its controlling promoter, and wherein the second coding region of the gene is in antisense orientation relative to its controlling promoter. If transcription of the sense and antisense coding regions of the targeted portion of the targeted gene occurs from two separate promoters, the result may be two separate RNA strands that may subsequently anneal to form a gene-inhibitory siRNA used to practice methods as provided herein.

In another aspect, transcription of the sense and antisense targeted portion of the targeted gene is controlled by a single promoter, and the resulting transcript will be a single hairpin RNA strand that is self-complementary, i.e., forms a duplex by folding back on itself to create a gene-inhibitory siRNA molecule. In this configuration, a spacer, e.g., of nucleotides, between the sense and antisense coding regions of the targeted portion of the targeted gene can improve the ability of the single strand RNA to form a hairpin loop, wherein the hairpin loop comprises the spacer. In one embodiment, the spacer comprises a length of nucleotides of between about 5 to 50 nucleotides. In one aspect, the sense and antisense coding regions of the siRNA can each be on a separate expression vector and under the control of its own promoter.

Inhibitory Ribozymes

In alternative embodiment, compositions and methods as provided herein comprise use of ribozymes capable of binding and inhibiting, e.g., decreasing or inhibiting, expression of one or a set of MMP transcripts or proteins, or isoforms or isoforms thereof, including e.g., MMP-1, MMP-2, MMP-3, MMP-9, MMP-11, and/or MMP-13 or any combination thereof, optionally in a liver-specific or liver-selective manner.

These ribozymes can inhibit a gene's activity by, e.g., targeting a genomic DNA or an mRNA (a message, a transcript). Strategies for designing ribozymes and selecting a gene-specific antisense sequence for targeting are well described in the scientific and patent literature, and the skilled artisan can design such ribozymes using these reagents. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

### Pharmaceutical Compositions and Formulations

In alternative embodiments, provided are pharmaceutical compositions and formulations for practicing methods as provided herein, e.g., methods for enhancing or accelerating liver regeneration, optionally enhancing or accelerating liver regeneration after tissue injury or liver resection; enhancing or accelerating liver repair, optionally enhancing or accelerating liver repair after a trauma, an injury or an infection, wherein optionally the injury is an ischemia-reperfusion injury; or, reducing the extent of or abolishing ischemia-reperfusion injury in a normal liver or a fatty liver, or a cadaver or donor liver or transplant liver, optionally in an individual in need thereof, optionally in a liver-specific or liver-selective manner.

In alternative embodiments, compositions used to practice the methods as provided herein are formulated with a pharmaceutically acceptable carrier. In alternative embodiments, the pharmaceutical compositions used to practice the methods as provided herein can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents used to practice the methods as provided herein can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions used to practice the methods as provided herein include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations used to practice the methods as provided herein can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, geltabs, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations used to practice the methods as provided herein can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a composition used to practice the methods as provided herein) in admixture with excipients suitable for the manufacture of aqueous suspensions. Such excipients include a suspending agent, such as sodium carboxymethylcellulose, methylcellulose, hydroxypropyl-methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration hydrophobic active agents used to practice the methods as provided herein. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations as provided herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing methods provided herein, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing methods provided herein, the pharmaceutical compounds can be delivered by transdermally, by a topical route, formulated as applicator sticks, solutions, suspensions, emulsions, gels, creams, ointments, pastes, jellies, paints, powders, and aerosols.

In practicing methods provided herein, the pharmaceutical compounds can also be delivered as nanoparticles or microspheres for regulated, e.g., fast or slow release in the body. For example, nanoparticles or microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674. Nanoparticles can also be given intravenously, for example nanoparticles with linkage to biological molecules as address tags could be targeted to specific tissues or organs.

In practicing methods provided herein, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations used to practice the methods as provided herein can be lyophilized. Provided are a stable lyophilized formulation comprising a composition as provided herein, which can be made by lyophilizing a solution comprising a pharmaceutical as provided herein and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations used to practice the methods as provided herein can be delivered by the use of liposomes or nanoliposomes. By using liposomes, particularly where the liposome surface carries ligands specific for target cells, e.g., liver cells, or are otherwise preferentially directed to a specific organ or tissues, e.g., liver, a heart, a kidney, muscle, bone, skin, trachea, arterial or venous blood vessels, intestine, spinal cord, nerve or a brain, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations used to practice the methods as provided herein can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions as provided herein are administered in an amount sufficient to for e.g., enhancing or accelerating liver regeneration, optionally enhancing or accelerating liver regeneration after tissue injury or liver resection; enhancing or accelerating tissue or organ repair, optionally enhancing or accelerating tissue or organ repair after a trauma, an injury or an infection, wherein optionally the injury is an ischemia-reperfusion injury, e.g., a heart attack or a stroke; or, reducing the extent of or abolishing ischemia-reperfusion injury in a tissue or organ, e.g., a normal liver or a fatty liver, in an individual, or in a cadaver or donor tissue or organ or transplant tissue or organ, e.g., or a cadaver or donor heart, lung, kidney, skin, or pancreas intended for transplant, in need thereof.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration of compositions used to practice the methods as provided herein can be in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ or tissue. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, e.g., compositions for treating liver or other infections (e.g., hepatitis), liver cirrhosis, liver or other cancers, septic shock, fever, pain and related symptoms or conditions. For example, the methods and/or compositions and formulations as provided herein can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Nanoparticles, Nanolipoparticles and Liposomes

Also provided are nanoparticles, nanolipoparticles, vesicles and liposomal membranes comprising compounds used to practice the methods as provided herein, e.g., to deliver compositions used to practice methods as provided herein (e.g., MMP inhibitors) to mammalian, e.g., liver, cells, or liver tissue, in vivo, in vitro or ex vivo. In alternative embodiments, these compositions are designed to target specific molecules, including biologic molecules, such as polypeptides, including cell surface polypeptides, e.g., for targeting a desired cell type, e.g., a liver cell, or a liver endothelial or sinusoidal cell, and the like.

Provided are multilayered liposomes comprising compounds used to practice methods as provided herein, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, e.g., as described in Park, et al., U.S. Pat. Pub. No. 20070042031, including method of producing a liposome by encapsulating an active agent (e.g., MMP-inhibiting nucleic acids and polypeptides), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, e.g., for targeting delivery of a compound (e.g., MMP-inhibiting nucleic acid or polypeptide) used to practice methods as provided herein to a desired cell type (e.g., a liver endothelial cell, a liver sinusidal cell, or any liver tissue in need thereof), as described e.g., in U.S. Pat. Pub. No. 20070110798.

Provided are nanoparticles comprising compounds (e.g., MMP-inhibiting nucleic acids and polypeptides) used to practice methods as provided herein in the form of active agent-containing nanoparticles (e.g., a secondary nanoparticle), as described, e.g., in U.S. Pat. Pub. No. 20070077286. In one embodiment, provided are nanoparticles comprising a fat-soluble active agent used to practice a method as provided herein or a fat-solubilized water-soluble active agent to act with a bivalent or trivalent metal salt.

In one embodiment, solid lipid suspensions can be used to formulate and to deliver compositions used to practice methods as provided herein to mammalian, e.g., liver, cells in vivo, in vitro or ex vivo, as described, e.g., in U.S. Pat. Pub. No. 20050136121.

### Delivery Vehicles

In alternative embodiments, any delivery vehicle can be used to practice the methods as provided herein, e.g., to deliver compositions methods as provided herein (e.g., MMP inhibitors) to mammalian, e.g., human, liver cells in vivo, in vitro or ex vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex is used to formulate a composition used to practice a method as provided herein, e.g. as described, e.g., in U.S. Pat. Pub. No. 20040151766.

In one embodiment, a composition used to practice methods as provided herein can be applied to cells using vehicles with cell membrane-permeant peptide conjugates, e.g., as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, e.g., using any electroporation system as described e.g. in U.S. Pat. Nos. 7,109,034; 6,261,815; 5,874,268.

### Products of Manufacture and Kits

Provided are products of manufacture and kits for practicing methods as provided herein, e.g., for: enhancing or accelerating liver regeneration, optionally enhancing or accelerating liver regeneration after tissue injury or liver resection; enhancing or accelerating liver repair, optionally enhancing or accelerating liver repair after a trauma, an injury or an infection, wherein optionally the injury is an ischemia-reperfusion injury; or, reducing the extent of or abolishing ischemia-reperfusion injury in a normal liver or a fatty liver, or a cadaver or donor liver or transplant liver.

The invention will be further described with reference to the examples described herein; however, it is to be understood that the invention is not limited to such examples.

### EXAMPLES

### Example 1: Liver-Selective MMP-9 Inhibition in the Rat Eliminates Ischemia-Reperfusion Injury and Accelerates Liver Regeneration

This example demonstrates that liver-selective MMP-9 inhibition can be a therapeutic tool for liver injury that damages the vasculature, whereas systemic matrix metalloproteinase inhibition can enhance the benefit of stem cell therapy with endothelial progenitor cells.

Recruitment of liver sinusoidal endothelial cell progenitor cells, so-called sprocs, from the bone marrow by VEGF-sdf1 signaling promotes recovery from injury and drives liver regeneration. Matrix metalloproteinases (MMPs) can proteolytically cleave VEGF, which might inhibit progenitor cell recruitment, but systemic matrix metalloproteinase inhibition might prevent efflux of progenitors from the bone marrow. The hypothesis for this study was that liver-selective MMP-9 inhibition would protect the hepatic VEGF-sdf-1 signaling pathway, enhance bone marrow sproc recruitment, and thereby ameliorate liver injury and accelerate liver regeneration, whereas systemic MMP inhibition would impair bone marrow sproc mobilization and therefore have less benefit or would be detrimental. Results: Liver-selective MMP-9 inhibition accelerated liver regeneration after partial hepatectomy by 40%, whereas systemic MMP inhibition impaired liver regeneration. Liver-selective MMP-9 inhibition largely abolished warm ischemia-reperfusion injury. In the extended hepatectomy model, liver-selective MMP-9 inhibition restored liver sinusoidal endothelial cell integrity, enhanced liver regeneration, and reduced ascites. Liver-selective MMP-9 inhibition markedly increased recruitment and engraftment of bone marrow sprocs, whereas systemic MMP inhibition impaired mobilization of bone marrow sprocs and their hepatic engraftment. Hepatic MMP-9 proteolytically cleaved VEGF after partial hepatectomy. Liver-selective MMP-9 inhibition prevented VEGF cleavage and doubled protein expression of VEGF and its downstream signaling partner sdf-1. In contrast, systemic MMP inhibition enhanced recruitment and engraftment of infused allogeneic progenitors. Conclusion: Liver-selective MMP inhibition prevents proteolytic cleavage of hepatic VEGF, which enhances recruitment and engraftment of bone marrow sprocs after liver injury. This ameliorates injury and accelerates liver regeneration.

After various forms of liver injury, hepatic VEGF is the central mediator that signals through stromal cell derived factor-1 (sdf1 or CXCL12) to induce proliferation in the bone marrow, mobilization to the circulation, and engraftment in the liver of CXCR7+ liver sinusoidal endothelial cell progenitor cells (sprocs) and signals through the nitric oxide pathway to induce the differentiation of engrafted sprocs to fenestrated liver sinusoidal endothelial cells (LSECs) (1-3). Although there is ample evidence that LSECs drive liver regeneration (4-6), our group has shown that this function is fulfilled by engrafted BM sprocs amongst the LSECs. Bone marrow suppression impairs liver regeneration after partial hepatectomy and the effect of bone marrow suppression is fully offset by infusion of whole bone marrow or of sprocs (1-3). Only the CXCR7+ subset of circulating sprocs engraft in the liver (1) and knockout of CXCR7+ endothelial cells in the body impairs liver regeneration (7). In addition to driving liver regeneration, recruitment of BM sprocs promotes recovery from toxic injury (2, 8).

There are two reasons to assume sprocs are a subset of endothelial progenitor cells. First, after partial hepatectomy there is a two-fold increase in circulating sprocs that persists for 72 hours (3). At 6 hours after partial hepatectomy, upwards of 90% of these cells are the CXCR7⁺ fraction that engrafts in the liver (1), but from 24 to 72 hours the circulating endothelial progenitor cells are almost all CXCR7⁻. Second LSECs are CD31+ (a classic endothelial marker) and CD45+ (8, 9) and we therefore define sprocs as progenitor cells with both these markers. Endothelial progenitor cells are often defined as the CD45− fraction of circulating progenitor cells. This further supports that LSEC progenitor cells or sprocs are a sub-set of endothelial progenitor cells.

Disparate types of injury that target LSECs increase matrix metalloproteinase (MMP) activity, including cold preservation injury (10), sinusoidal obstruction syndrome (11), acetaminophen toxicity (12), ischemia-reperfusion injury (13, 14), and small-for-size syndrome (15). The literature has conflicting reports on whether MMPs enhance [to cite just a few (16-18)] or reduce (19) VEGF effects. An explanation for MMP-mediated reduction of VEGF activity is the finding that MMPs, including MMP-9, can proteolytically cleave VEGF₁₆₄ (19), generating VEGF with a dysfunctional angiogenic response. This suggests that inhibition of hepatic MMP activity after liver injury might enhance VEGF-sdf1 recruitment and repair by BM sprocs. Conversely, proteolytic cleavage by MMPs, including MMP-9, of extracellular matrix and cytokines that retain stem cells in their niche is necessary for mobilization of BM stem cells (20, 21). Thus, inhibition of hepatic MMP activity might improve BM sproc recruitment and engraftment in the liver, whereas inhibition of BM MMP should impair BM sproc mobilization and diminish repair of liver injury.

Based on the above, we formulated the following two hypotheses. 1. Liver-selective inhibition of MMP with sparing of BM MMP should reduce liver injury and promote liver regeneration, whereas systemic MMP inhibition should be less beneficial or even detrimental. 2. Engraftment of infused sprocs for stem cell therapy should benefit from systemic MMP inhibition by preventing proteolytic cleavage of hepatic VEGF, and by reducing release of sprocs from the BM and thereby decreasing competition for engraftment.

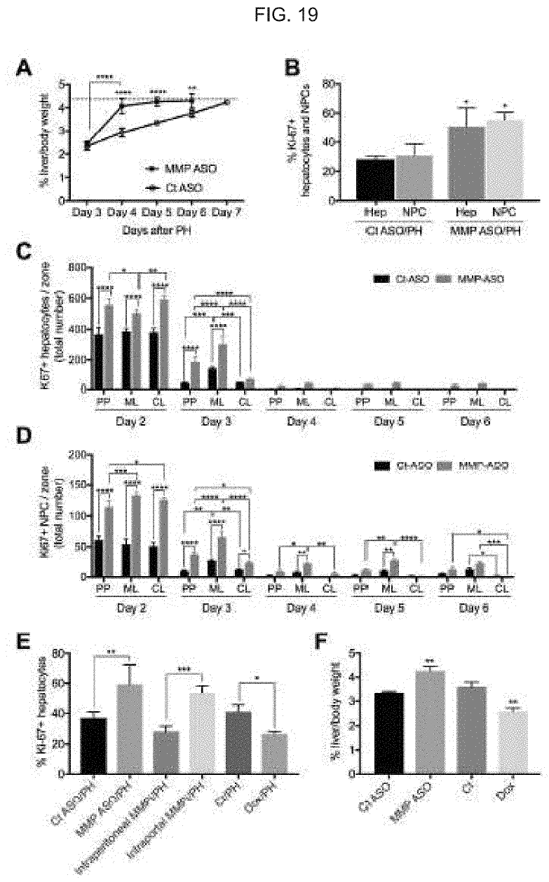

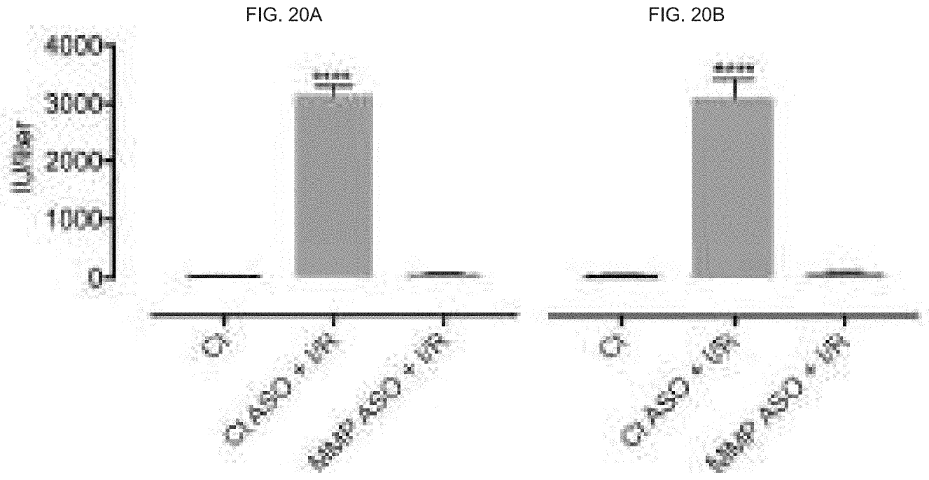

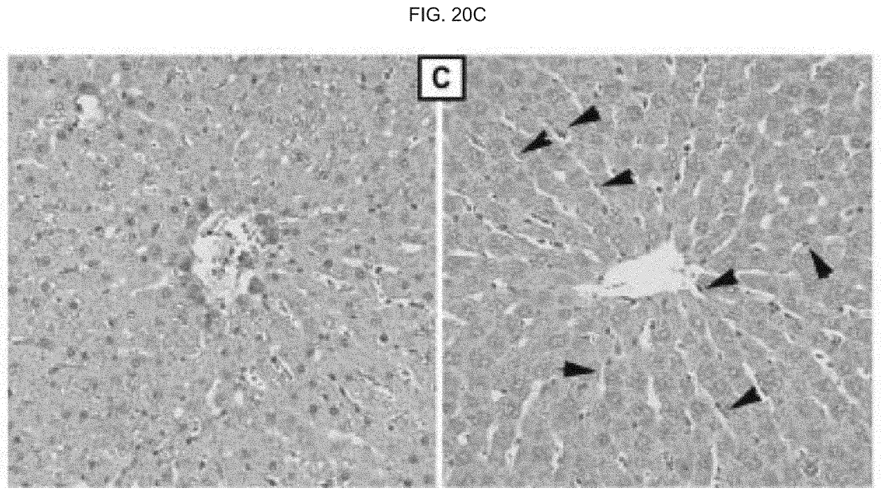

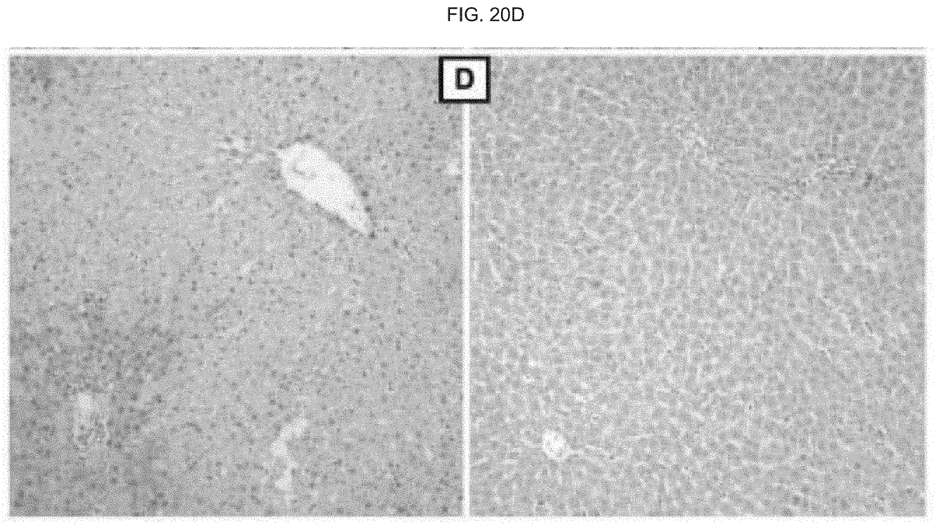

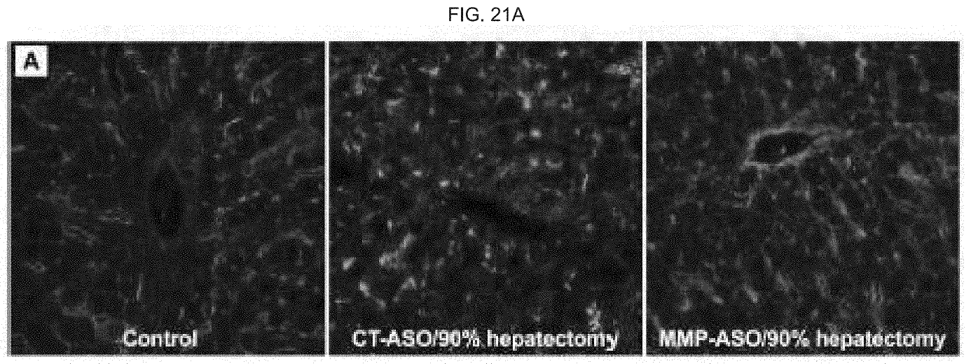

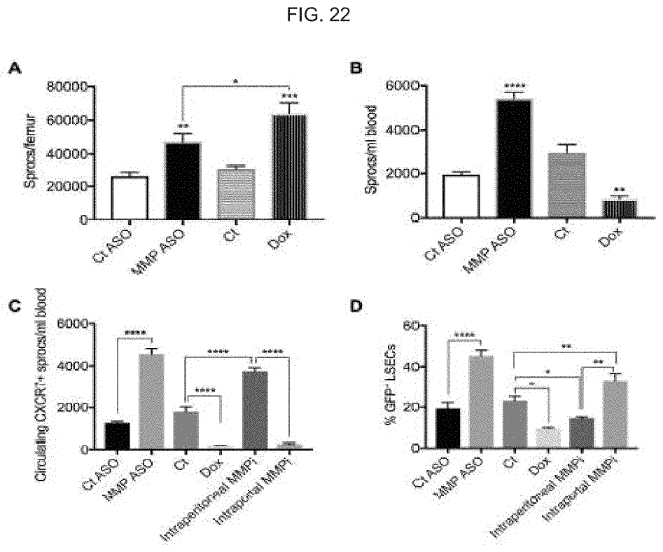

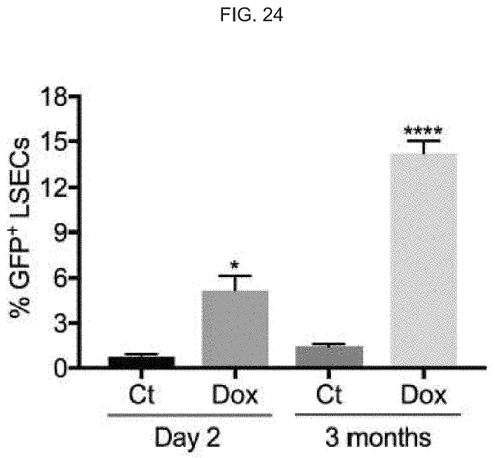

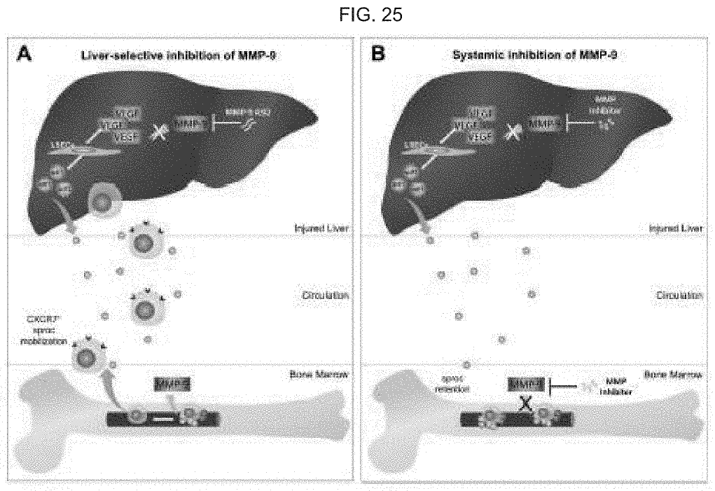

The therapeutic benefit of liver-selective MMP-9 inhibition is shown in models of two-thirds partial hepatectomy (PH) (FIG. 19), ischemia-reperfusion injury (FIG. 20), and 90% hepatectomy as a model of small-for-size syndrome (FIG. 21). Examination of the mechanisms by which liver-selective MMP-9 inhibition protects against injury and promotes liver regeneration are shown in FIG. 22 and FIG. 23. The use of systemic MMP inhibition to promote engraftment of infused sprocs, as a model for stem cell therapy, is shown in FIG. 24. FIG. 25A-B is a diagram that describes the molecular pathways underlying the mechanisms.

### Material and Methods

Reagents. Chemicals were obtained from Sigma-Aldrich unless stated otherwise.

Animal studies. Lewis rats were obtained from Harlan (Placentia, Calif.). Breeding pairs of Lew-Tg(CAG-EGFP)ys rats were obtained from the National Institutes of Health Rat Resource and Research Center at the University of Missouri. Male rats were used for the experiments. Rats were kept in conventional housing for rats, consisting of Allentown polycarbonate rat cages with filter tops and Sani-Chips bedding (wood product), in a 12:12-h light-dark cycle (lights on from 6 AM to 6 PM) at room temperature (21-23° C.), with 5 μm-filtered water delivered to cages via an Edstrom automatic watering valve. Purina Lab Diet 5001 and water were provided ad libitum. Rats that showed distress postoperatively, based on activity level, behavior, appearance, reduced intake of food or water, or respiratory distress, were euthanized.

All protocols were reviewed and approved by the Animal Care and Use Committee at the University of Southern California to ensure ethical and humane treatment of the animals. This study followed the guidelines outlined in the Office of Laboratory Animal Welfare “Public Health Service Policy on Humane Care and Use of Laboratory Animals” (2015).

Partial (70%) hepatectomy was performed under general anesthesia with ketamine-xylazine (80-90 mg/kg ip). For 70% partial hepatectomy, the median and left lateral lobes were removed. Buprenorphine SR (1 mg/kg) was used postoperatively for analgesia.

Extended hepatectomy. To achieve 90% hepatectomy, the medial and left lateral, and the superior and inferior portion of the right lobe were resected with preservation only of the caudate lobe. To prevent post-operative hypoglycemia, animals were gavaged once with 0.5 ml 20% glucose and for the first 16 hours 20% glucose was used as oral hydration that was provided through a liquid diet feeding tube (Biosery cat 9010) that was positioned so rats could drink without significant exertion, followed by free access to water. Rats received standard laboratory chow ad libitum. All rats survived until they were euthanized on day 2.

Ischemia-reperfusion model. Ischemia was induced by clamping the vessels in the hilum that perfuse the medial and left lateral lobes with a nontraumatic microvascular clip. After 1 hour, the microvascular clip was released. The abdomen was closed and the rats were left to recover with free access to standard chow diet and water ad libitum. Preliminary studies established that ALT and AST peaked at 6 hours.

Bone marrow transplantation. BM cells were obtained from one tibia and femur from the donor. Recipients underwent 1,000 cGy total body irradiation and were injected via tail vein with 50 million BM cells. Rats received oxytetracycline (200 mg/ml) diluted 1:1,000 in the drinking water starting two days before irradiation and continuing until one week after irradiation. BM was allowed to engraft for 2 months before use. BM from Lew-Tg(CAG-EGFP)ys Lewis rats was transplanted into wild-type Lewis rats and cells were tracked by GFP expression.

Liver-selective MMP-9 inhibition: Knockdown of MMP-9 was achieved by injection of one of two MMP-9 antisense oligonucleotides that were a kind gift from Ionis Pharmaceuticals (Ions No 283953 and 283973, Carlsbad, Calif.; 20 mg/kg intraperitoneally twice weekly for 4 weeks.

Hepatic MMP-2/9 inhibition in other studies was performed by infusing 2-[(4-biphenylsulfonyl)amino]-3-phenyl-propionic acid (Abcam, Cambridge, Mass.), 100 μg/h, into the portal circulation by an Alzet mini-osmotic pump (model 2ML 1; Alza Corporation, Palo Alto, Calif.) via a cannula inserted into the inferior mesenteric vein starting 2 days before the relevant study.

Systemic MMP inhibition: Doxycycline (Sigma, St. Louis), 15 mg/kg i.g., was given twice daily starting 2 days before the relevant study. Control studies were performed with the same dose of a chemically modified tetracycline, isochlorotetracycline, which is only a weak MMP inhibitor. In other experiments, MMP-2/9 was inhibited by infusing 2-[(4-biphenylsulfonyl)amino]-3-phenyl-propionic acid, 100 μg/kg/h, intraperitoneally with an Alzet mini-osmotic pump (model 2001, Durect Corporation, Cupertino Calif.).

LSEC isolation. LSECs were isolated by collagenase perfusion, iodixanol density gradient centrifugation, and centrifugal elutriation, as previously described (22, 23). Yields averaged 86×10⁶ cells per normal rat liver (range 69-95×10⁶) with average viability of 94% (range 91-96%). Purity of the cells was 99%, as determined by uptake of formaldehyde-treated serum albumin (kind gift from Bard Smedsrod, University of Tromso, Tromso, Norway), a function specific to LSECs. Cells isolated by this protocol have an appropriate range of fenestrae organized in sieve plates.

Sproc isolation. BM and circulating sprocs were isolated by immunomagnetic selection for CD133 using a CD133 Cell Isolation Kit (Miltenyi Biotec, Auburn, Calif.) and separation with an autoMACS Pro (Miltenyi Biotec, Auburn, Calif.), followed by FACS sorting for CD45 and CD31 (FIG. 26, or Supporting Fig S1).

To isolate resident sprocs, LSEC were isolated as described above and the CD133+ fraction was obtained by immunomagnetic selection (3).

To determine the number of sprocs in the circulation and BM from Lewis rats 6 hours after partial hepatectomy, CD133+ cells were isolated by immunomagnetic selection for CD133 from peripheral blood or BM. Cell suspensions were preblocked with FcR blocking reagent and incubated for 60 minutes with antibodies to rat CD45 and CD31. CD133+45+31+ cells were quantified by flow cytometry. In some experiments cells were also stained with antibody to rat CXCR7, to identify CD133+45+31+CXCR7+ cells in the circulation.

Flow cytometry. Flow cytometry was performed using a FACSCalibur (BD Biosciences). Isotype control antibodies were used to determine appropriate gates, voltages, and compensation required for multivariate flow cytometry. Cell Quest Pro software was used for analysis

Immunohistochemistry for Ki-67

Deparaffinized 5 μm sections of liver were treated with Bond ER-2 antigen retrieval (Leica Biosystems, Buffalo Grove, Ill.) for 20 min. After preblocking for 10 min with Rodent Block R (Cat #RBR962G, Biocare, Pacheco, Calif.), sections were incubated with antibody against Ki-67 for 60 min. Immunohistochemistry was performed by incubating sections with rabbit-on-rodent HRP-Polymer (cat #RMR622G, Biocare) for 30 min. Slides were rinsed in Bond diaminobenzidine (Leica Biosystems) for 10 min. After counterstaining with Bond hematoxylin, slides were dehydrated and covered using a coverslip with resinous mounting medium (Leica Biosystems).

Ki-67 positive cells were determined using photographs taken with a 20×-objective in 15 lobules per rat, n=3. To assess the percentage of proliferating hepatocytes and non-parenchymal cells, slides were coded and the percentage Ki-67 positive cells was determined blindly by X.W. To determine zonation of proliferating cells, each lobule was divided into three even fields and the total number of ki-67 positive cells/field was counted.

Measurements of serum ALT and AST were performed by IDTOX Alanine Transaminase (ALT) Color Endpoint Assay kit (cat #sup 6001-c, Empire genomics, Buffalo, N.Y.) and IDTOX Aspartate Transaminase (AST) Color Endpoint Assay kit (cat #sup 6002-c, Empire genomics).

CD31 staining. Frozen sections of liver were fixed in cold acetone and stained with a mouse monoclonal anti-CD31 and m-IgG-kappa BP-FITC anti-mouse IgG. Slides were examined using a Nikon PCM-2000 confocal microscope with a 488-nm laser excitation wavelength.

Immunoblotting. Samples from freshly isolated liver were harvested using triple lysis buffer (TLB: 50 mM Tris base, 150 mM NaCl, 3 mM sodium azide, 12 mM sodium deoxycholate, 0.1% SDS, 1% Nonidet-P40) supplemented with 2 mM phenylmethylsulphonyl fluoride (PMSF), 1 mM sodium orthovanadate and 1% protease inhibitors cocktail (Santa Cruz sc-24948). Proteins were purified by centrifugation at 15,000 g at 4° C. for 10 min and quantified using DC™ Protein Assay Kit (BioRad). The samples obtained from bone marrow extracellular fluid were preserved in PBS. For MMP-9 analysis, 100 μg of total protein was used from each sample per lane and resolved on NUPAGE™ NOVEX™ 10% Bis-Tris Protein Gels using MOPS running buffer. Proteins were transferred onto 0.45 μm nitrocellulose membranes via electroblotting using XCell II™ Blot Module (Invitrogen), for 1 h, 30V. For SDF-1 and VEGF analysis, 75 μg of total protein was used from each sample per lane and resolved on NUPAGE™ NOVEX™ 4-12% Bis-Tris Protein Gels using MES running buffer. Proteins were transferred onto 0.2 μm nitrocellulose membranes via electroblotting using Trans-blot SD Semi-Dry Transfer Cell™ (Bio-rad), for 20 min, 25V. Membranes were blocked using NAP-BLOCKER™. Blots were probed with primary antibody overnight followed by IRDye secondary antibodies. Membrane digital images were acquired with the Odyssey Infrared Imaging System (LI-COR) and analyzed using the LI-COR IMAGE STUDIO™. Analyses were performed in triplicate and normalized by GAPDH or ß-actin values.

Statistical analysis. Unless stated otherwise, statistical analysis was performed by ANOVA and, if the ANOVA was statistically significant, analyzed post-hoc by Fisher's least significant difference using GRAPHPAD PRISM™. Levels of statistical significance are * p<0.05, ** p<0.01, *** p<0.001, and **** p<0.0001. Unless otherwise indicated, statistical significance is compared to the appropriate control. All experiments were performed with n=3 unless stated otherwise.

### Acceleration of Liver Regeneration

Pro-MMP-9 is stored in granules in cells and its proteolytic activity occurs extracellularly after degranulation and cleavage to MMP-9. In normal liver, LSECs are a major source of MMP-9 (FIG. 27 (Supporting Fig S2A)) which is consistent with the LSEC as the liver cell with the highest MMP-9 activity (11). PH increased MMP-9 expression in the liver and MMP-9 antisense oligonucleotides (ASO) abrogated the increase (FIG. 18A and FIG. 18B, and FIG. 28A-B (Supporting Fig S2B). Liver-selectivity of the ASO was confirmed by measuring MMP-9 activity in the extracellular fluid of the BM. BM MMP-9 activity was increased after PH, but MMP-9 ASO pretreatment did not alter BM MMP-9 activity (FIG. 18C and FIG. 18D, and FIG. 29A-B (Supporting Fig S2C).

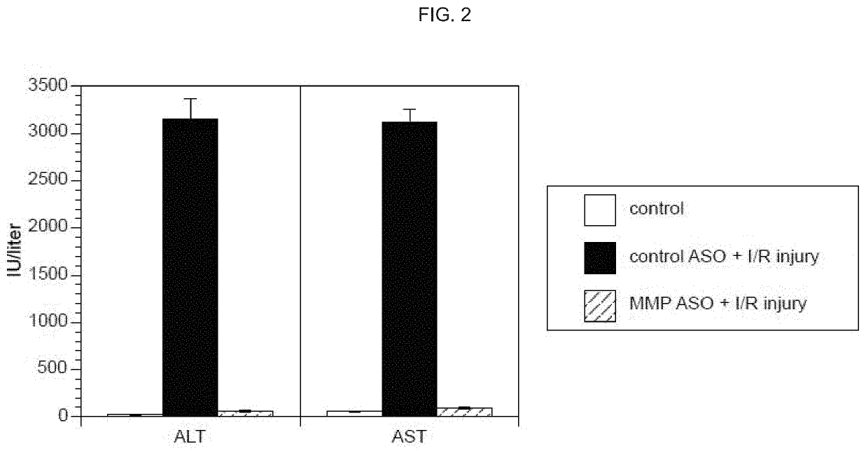

FIG. 19A (or FIG. 2A of Example 1) is a key panel. MMP-9 ASO accelerated liver regeneration by 40%; to the best of our knowledge, this degree of acceleration is unprecedented.

A time-course of liver-to-body-weight from day 3 to day 7 after PH demonstrates that the liver-to-body weight ratio reaches that of control littermates by day 4 after MMP-9 ASO pretreatment compared to day 7 in the control-ASO pretreated group. MMP-9 ASO pre-treatment increased proliferation of hepatocytes and non-parenchymal cells by 80% on day 2 (FIG. 19B, or FIG. 2B of Example 1), Hepatocyte proliferation in the MMP-9 ASO group is higher in all 3 zones of the liver on day 2 and higher in the periportal and midlobular region on day 3 (FIG. 19C, or FIG. 2C of Example 1). For non-parenchymal cells from rats treated with MMP-9 ASO, proliferation is higher in all 3 zones on day 2 and day 3 and proliferation is higher in the midlobular region on days 4 and 5 (FIG. 19D, or FIG. 2D of Example 1). Liver-selective MMP-9 inhibition with either MMP-9 ASO or intraportal MMP-2/9 inhibitor enhances hepatocyte proliferation on day 2 compared to their respective controls, whereas systemic MMP inhibition with doxycycline, which inhibits several MMPs including MMP-9 (24, 25), reduces hepatocyte proliferation compared to its solvent control (FIG. 19E, or FIG. 2E of Example 1). Of note, neither doxycycline nor intraportal MMP-2/9 inhibitor given to controls was hepatotoxic (FIG. 30A-B (Supporting Fig S3A)).

Comparison of liver-selective versus systemic MMP inhibition demonstrates that liver-to-body weight ratio on day 5 is increased in the MMP-9 ASO pretreatment group by 27% compared to control ASO pretreatment (FIG. 19F, or FIG. 2F of Example 1). Systemic MMP inhibition with doxycycline reduces liver-to-body weight ratio on day 5 by 29% compared to its solvent control (FIG. 19F, or FIG. 2F of Example 1). To rule out the possibility that liver-selective MMP inhibition recruited a stem cell for one of the other liver cell types, livers were digested on day 2 after PH and examined by flow cytometry. At least 94% of CD133+ cells were CD31+ cells. i.e. endothelial cells (FIG. 31 (Supporting Fig S3B)).

### Prevention of Ischemia/Reperfusion (FR) Injury

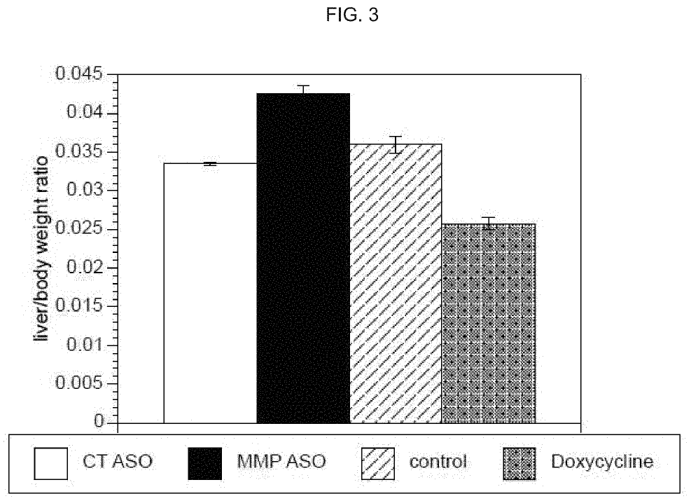

Induction of ischemia for 1 hour, followed by 6 hours of reperfusion lead to extremely high elevations of ALT and AST (FIG. 20A and FIG. 20B, or FIGS. 3A and 3B of Example 1). Pre-treatment with MMP-9 ASO largely abolished injury as indicated by ALT (58 IU/l±10) and AST (95 IU/l±12) in the near-normal or modestly elevated range, respectively (FIG. 20A and FIG. 20B, or FIGS. 3A and 3B of Example 1). Histology (FIG. 20C and FIG. 20D, or FIGS. 3C and 3D of Example 1) showed clearing of hepatocyte cytoplasm, lobular disarray, and absence of LSECs in the control-ASO/FR injury group (left panels), whereas the MMP-9 ASO/FR injury group showed preservation of hepatocyte integrity and presence of LSECs (right panels). The findings suggest that the predominant injury at 6 hours was to LSECs rather than to hepatocytes: LSECs were largely absent in the control ASO pretreated group (FIG. 20C, or FIG. 3C of Example 1), but there was only clearing of cytoplasm and no frank necrosis of hepatocytes. Thus pretreatment by MMP-9 ASO prevented subsequent hepatocyte necrosis by promoting repair of the LSEC lining.

### Attenuation of Small-for-Size Syndrome

In living-donor related transplantation or after extensive liver resection for metastases, the recipient's existing portal vein flow does not adapt to the small liver. Damage to the liver graft or remnant is thought to be due to hyper-perfusion and the injury is known as small-for-size syndrome. Extended hepatectomy, removal of 90% of the liver, is a model for small-for-size syndrome. On day 2 after extended hepatectomy, CD31 staining demonstrated a marked absence of LSECs lining the sinusoids (FIG. 21, or FIG. 4A of Example 1, middle panel), which is consistent with collapse of sinusoids seen during the first 72 hours after PH (26). In contrast, the sinusoids of rats pre-treated with MMP-9 ASO followed by extended hepatectomy (FIG. 21A, or FIG. 4A of Example 1, right panel) had LSEC lining that appears comparable to the untreated control livers (FIG. 21A, or FIG. 4A of Example 1, left panel). The 3 panels of FIG. 21A, or FIG. 4A are centered on the pericentral lobule for the sake of comparison, but the respective changes in LSEC lining in the pericentral lobule were representative of the whole liver (data not shown). Pretreatment with MMP-9 ASO also reduced ascites on day 2 by 57% compared to control ASO pretreatment (FIG. 21C, or FIG. 4C of Example 1), likely due to patent sinusoids that improved liver hemodynamics.

### Increased Recruitment and Engraftment of BM Sprocs

To examine the mechanism of the therapeutic benefit of liver-selective MMP inhibition, the respective effects of liver-selective and systemic MMP inhibition on BM sproc recruitment and engraftment were examined in the PH model. MMP-9-ASO/PH increased the number of sprocs in the BM compared to control ASO/PH (FIG. 22A, or FIG. 5A of Example 1). Systemic MMP inhibition in the doxycycline/PH group increased the number of sprocs in the BM significantly more than MMP-ASO/PH, consistent with impairment of sproc release by MMP inhibition of the BM.

Mobilization: MMP-9-ASO/PH increased BM sproc mobilization to the circulation compared to ASO control/PH by 180%, whereas doxycycline/PH reduced mobilization of BM sprocs by 72% (FIG. 22B, or FIG. 5B of Example 1). This is consistent with the hypothesis that liver-selective MMP-9 inhibition protects the chemoattractant signaling to BM sprocs and promotes recruitment of sprocs from the BM, whereas systemic MMP inhibition prevents release of sprocs from the BM. BM sprocs that engraft in the liver are CXCR7+(1), so addition of this marker is a closer approximation of actual sprocs, i.e. LSEC-specific endothelial progenitors cells in the circulation. Liver-selective MMP inhibition with either MMP-9-ASO or with slow infusion into the portal circulation (intraportal infusion) of an MMP-2/9 inhibitor, (2R)-2-[(4-Biphenylylsulfonyl)amino]-3-phenylpropionic acid, increased CXCR7+ sprocs in the circulation by 260 and 110%, respectively, after PH, whereas systemic MMP inhibition with either doxycline or intraperitoneal injection of the MMP-2/9 inhibitor reduced the number of CXCR7+ sprocs mobilized to the circulation by 90 and 86%, respectively (FIG. 22C, or FIG. 5C of Example 1). Pharmacological inhibition with the MMP-2/9 inhibitor abrogated the increase in hepatic MMP-9 expression when given either liver-selectively by intraportal infusion or intraperitoneally (FIG. 18A, or FIG. 1A of Example 1).

Engraftment: To track BM sproc engraftment in the liver, wild type rats underwent BM transplantation with BM from transgenic EGFP+ rats. Liver-selective MMP inhibition with either MMP-9 ASO/PH or intraportal MMP-2/9 inhibitor/PH increased hepatic engraftment of GFP+BM sprocs by 134% and 45%, respectively, whereas systemic inhibition with either doxycycline/PH or intraperitioneal MMP-2/9 inhibition/PH reduced hepatic engraftment of BM sprocs by 58% and 36%, respectively, compared to control/PH (FIG. 22D, or FIG. 5D of Example 1). As a control, the effect of a different MMP-9 ASO on engraftment was examined and this had near-identical effect on engraftment to the MMP-ASO described above (FIG. 32, or Supporting Fig S4). To confirm that the effect of doxycycline was due to MMP inhibition rather than an antibiotic effect, isochlorotetracycline was administered prior to PH. Isochlorotetracycline had no effect on engraftment (FIG. 33).

### Mechanism of Liver-Selective MMP Inhibition

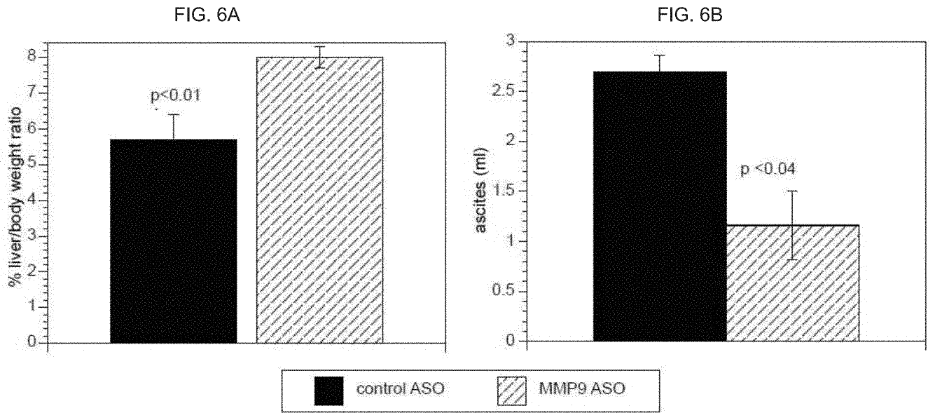

Upregulation of MMP-9 expression after PH (FIG. 18A-B, FIG. 1A and B of Example 1) (27) was accompanied by increased expression of a 17 kDa VEGF cleavage fragment (FIG. 23A-B, or FIG. 6A and B of Example 1), which is consistent with previous reports of MMP-9 producing a VEGF₁₆₄ cleavage product that has dysfunctional angiogenic properties (19, 28). Pretreatment with MMP-9 ASO completely prevented increased expression of the 17 kDa cleavage product after PH and lead to significantly higher hepatic VEGF₁₆₄ and sdf1 in the MMP-9 ASO/PH group compared to the ASO Ct/PH group (FIG. 23A-D, or FIG. 6 A-D of Example 1). MMPs, including MMP-9, can also cleave sdf-1, leading to decreased chemoattraction. We observed faint expression of a 6 kDa band of sdf-1 that increased after PH, but MMP-9 ASO pretreatment did not diminish the size of the 6 kDa band (data not shown).

### Increased Engraftment of Infused Stem Cells

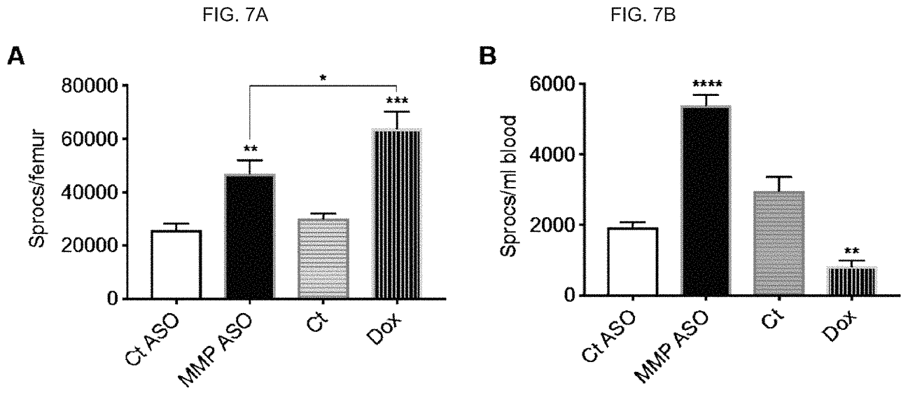

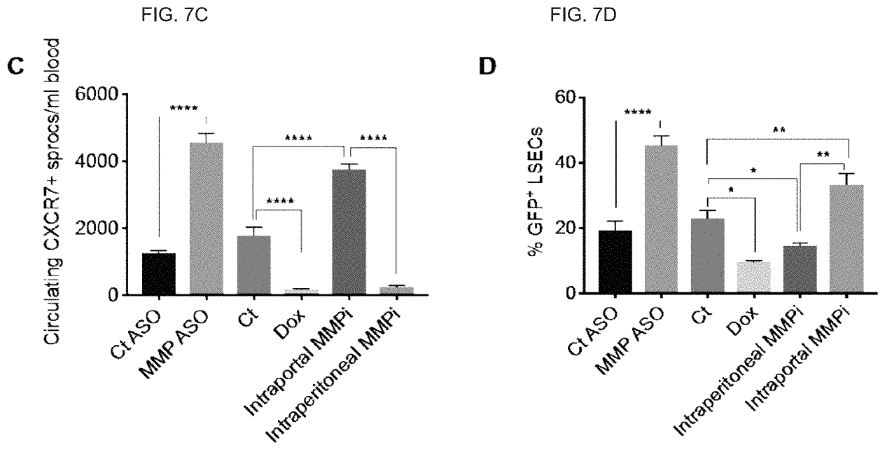

Stem cell therapy with infused cells requires engraftment. However progenitor cells that are infused after injury compete with BM progenitors. Indeed infusion of EGFP+ sprocs 6 hours after PH resulted in only 0.7% of LSECs derived from the infused sprocs on day 2 and 1.5% by 3 months (FIG. 24, or FIG. 7 of Example 1). Systemic inhibition of MMP with doxycycline, which reduces mobilization of BM sprocs (see above), increased engraftment of infused sprocs seven-fold by day 2 after infusion and almost ten-fold by 3 months (FIG. 24, or FIG. 7 of Example 1).

### Discussion

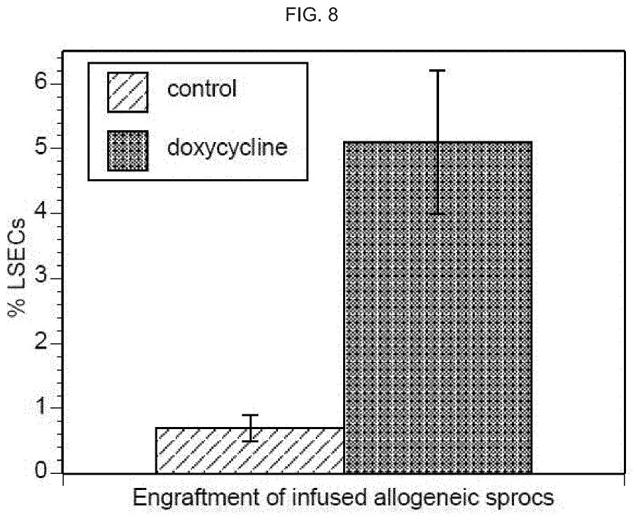

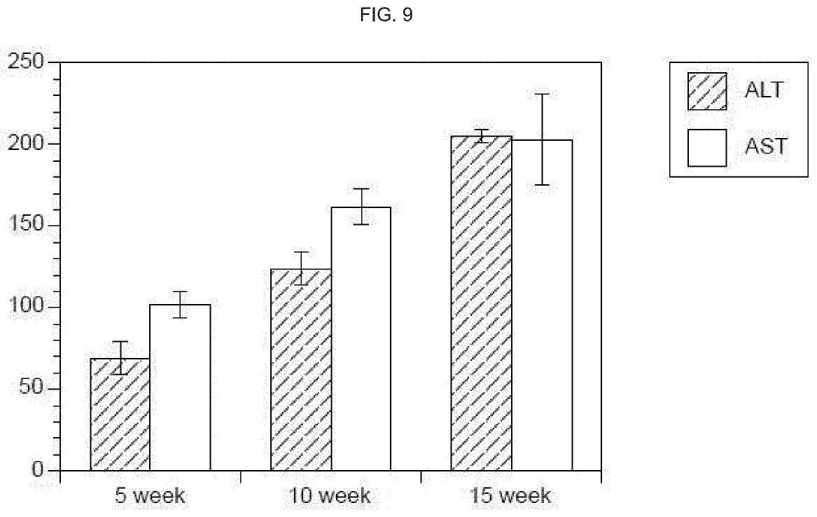

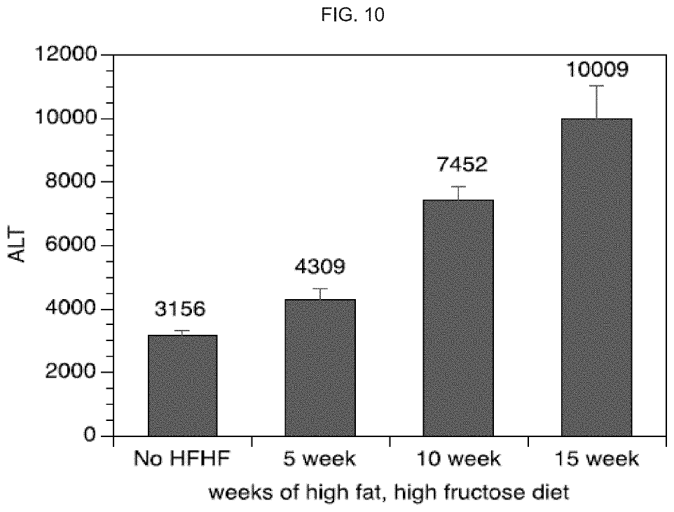

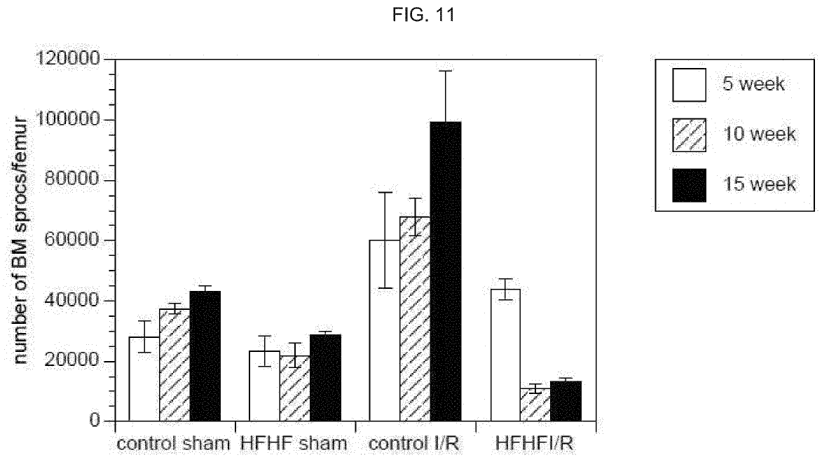

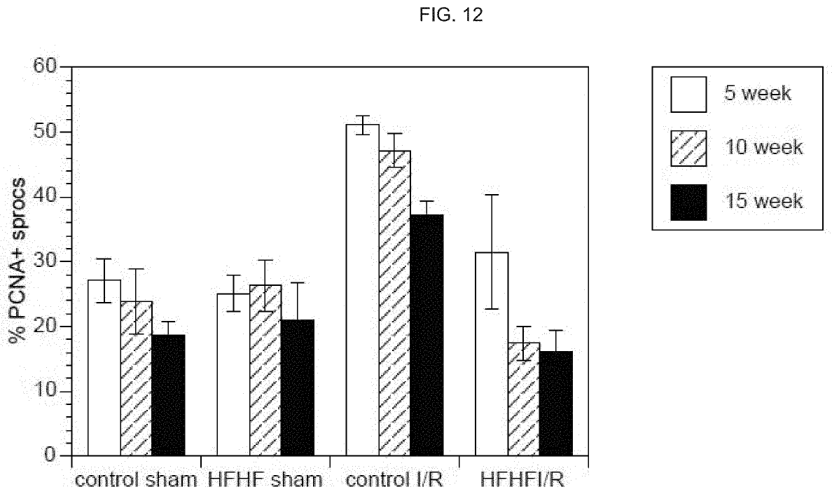

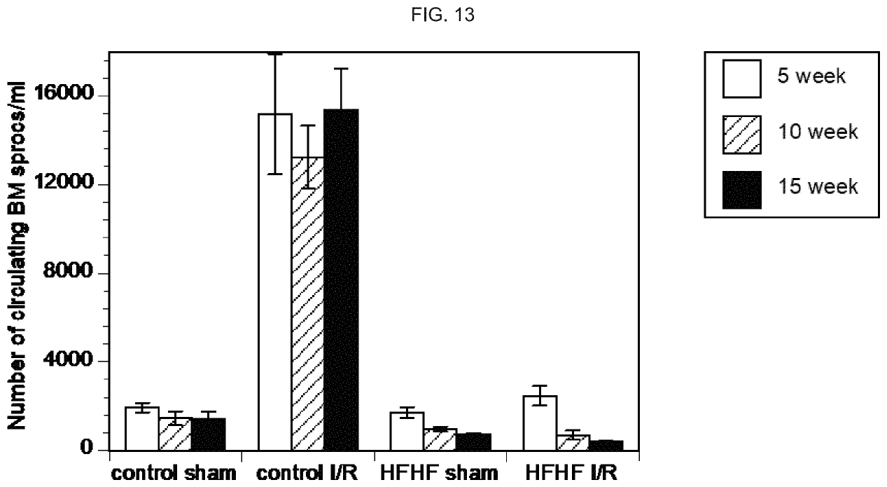

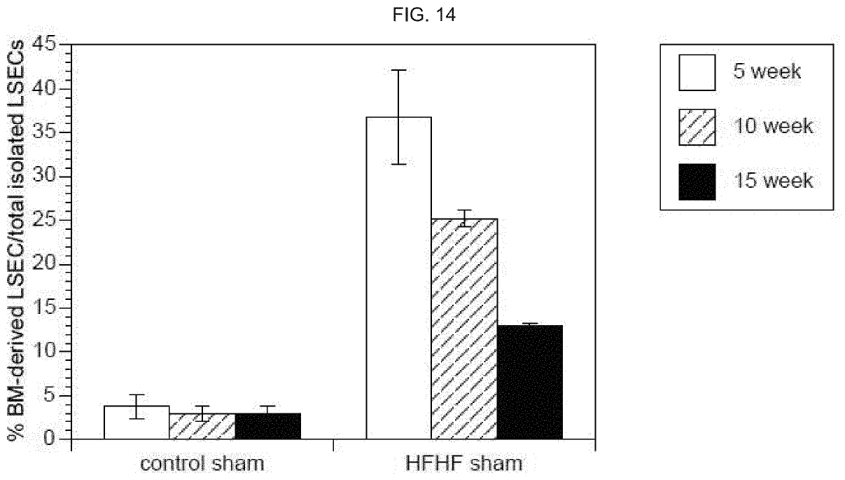

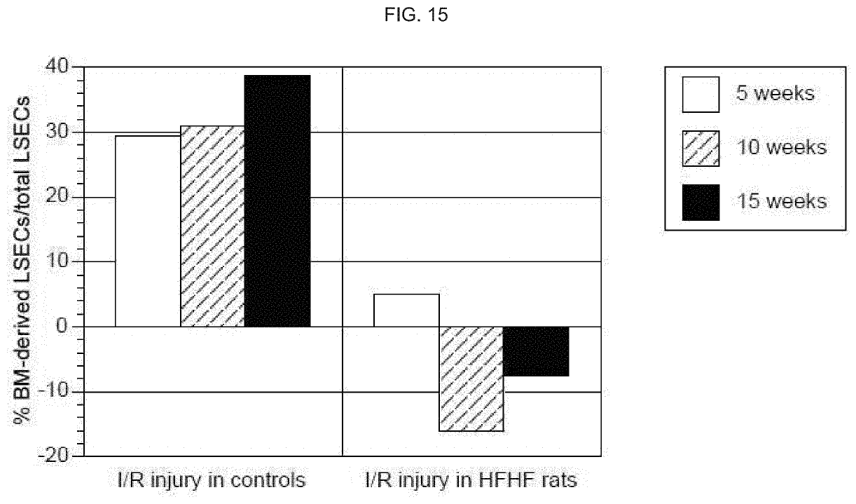

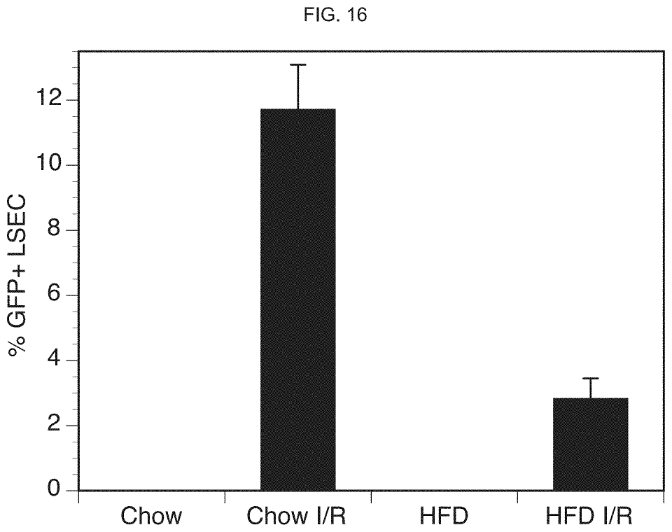

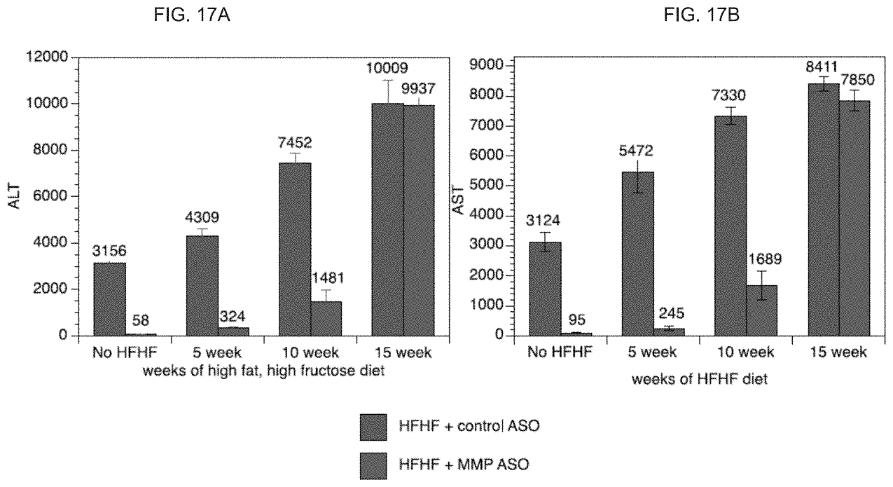

Liver-selective MMP inhibition accelerated liver regeneration after two-thirds PH by 40%, largely abolished FR injury, and accelerated liver regeneration and reduced ascites formation after extended hepatectomy. The mechanism is through inhibition of proteolytic cleavage by MMP-9 of VEGF₁₆₄, which resulted in increased expression of VEGF₁₆₄ and the downstream chemokine sdf-1. The VEGF-sdf1 pathway recruits BM endothelial progenitor cells of the LSECs, so-called BM sprocs, that are essential for liver regeneration (1, 2). In contrast, systemic MMP inhibition impaired recruitment of BM sprocs to the liver and reduced liver regeneration after PH by preventing mobilization of BM progenitor cells to the circulation. Consistent with this, systemic pharmacological inhibition of MMPs has been studied in liver injury models, but often with modest benefit (12, 13, 29, 30). FIG. 8 summarizes the findings described above.

Previous studies have demonstrated MMP-9 activity in LSECs but undetectable activity in hepatocytes, Kupffer cells, or hepatic stellate cells (11). Although antisense oligonucleotides have been designed that are more hepatocyte specific, the 2′-methoxyethyl ASOs used in the current study target both hepatocytes and non-parenchymal cells. Thus LSECs are the likely target of the MMP ASO used in the current study.

The findings reported here have translational implications for the liver. MMP inhibitors failed clinical trials to treat cancer, but good candidate drugs reportedly have no toxicity with short-term administration. Treatment of a cadaveric organ donor with systemic MMP inhibition might protect multiple organs from ischemia-reperfusion injury without inhibiting MMP in the recipients' BM. Serendipitous application of liver-selective MMP-2/9 inhibition completely prevented severe toxin-induced sinusoidal obstruction syndrome (11). The extended hepatectomy studies presented here suggest that liver-specific MMP inhibition would attenuate small-for-size syndrome. For living-related liver donor transplantation and for extended hepatectomy for liver tumors, MMP-9 inhibition would need to be strictly liver-specific to prevent MMP inhibition of the BM.

The basic mechanistic processes described here in liver injury mimic that found in other organs. MMPs increase after injury in several organs, including heart (31, 32), brain (33, 34), lung (35, 36), kidney (33, 37), and pancreas (38), and vascular wall (39). VEGF and sdf1 signaling recruit endothelial progenitor cells to the heart (40, 41), brain and spine (42-44), lung (45), kidney (46), peripheral nerves (47), bone (48, 49), and limbs (50). Thus the benefit of end-organ specific MMP inhibition is likely applicable to other organs and should apply to recovery from insults that injure the vasculature. The challenge will be to develop end-organ specific delivery of an MMP inhibitor, given the observation that systemic inhibition that inhibits BM MMP is detrimental. Slow infusion of MMP inhibitor into a specific organ may be an option if (near)-complete uptake of the MMP inhibitor by the organ can be achieved.

Stem cell therapy with infused cells requires engraftment and the current study demonstrates that engraftment of infused endothelial progenitor cells after injury is markedly increased by systemic MMP inhibition. One could envision that one application of this would be enhanced engraftment of endothelial progenitor cells that are gene-edited to be anticoagulant to reduce the risk of recurrent thrombosis or vascular occlusion in a compromised vascular bed.

### REFERENCES

1. Bo, C. J., et al. Effects of ischemic preconditioning in the late phase on homing of endothelial progenitor cells in renal ischemia/reperfusion injury. Transplant Proc 45, 511-516 (2013).
2. Kim, B. J., et al. Synergistic vasculogenic effects of AMD3100 and stromal-cell-derived factor-1alpha in vasa nervorum of the sciatic nerve of mice with diabetic peripheral neuropathy. Cell Tissue Res 354, 395-407 (2013).
3. Paczkowska, E., et al. Evidence for proangiogenic cellular and humoral systemic response in patients with acute onset of spinal cord injury. The journal of spinal cord medicine 38, 729-744 (2015).
4. Kawakami, Y., et al. SDF-1/CXCR4 axis in Tie2-lineage cells including endothelial progenitor cells contributes to bone fracture healing. Journal of bone and mineral research: the official journal of the American Society for Bone and Mineral Research 30, 95-105 (2015).
5. Anderson, E. M., et al. Local delivery of VEGF and SDF enhances endothelial progenitor cell recruitment and resultant recovery from ischemia. Tissue Eng Part A 21, 1217-1227 (2015).
6. Lu, A., Sun, B. & Qian, L. Combined iNO and endothelial progenitor cells improve lung alveolar and vascular structure in neonatal rats exposed to prolonged hyperoxia. Pediatr Res 77, 784-792 (2015).

Du, R., et al. Effect of integrin combined laminin on peripheral blood vessel of cerebral infarction and endogenous nerve regeneration. Journal of biological regulators and homeostatic agents 29, 167-174 (2015).
8. Herrmann, M., Verrier, S. & Alini, M. Strategies to Stimulate Mobilization and Homing of Endogenous Stem and Progenitor Cells for Bone Tissue Repair. Frontiers in bioengineering and biotechnology 3, 79 (2015).
9. Chang, T. T., et al. Direct Renin Inhibition with Aliskiren Improves Ischemia-Induced Neovasculogenesis in Diabetic Animals via the SDF-1 Related Mechanism. PLoS One 10, e0136627 (2015).
10. Chiang, K. H., et al. Statins, HMG-CoA Reductase Inhibitors, Improve Neovascularization by Increasing the Expression Density of CXCR4 in Endothelial Progenitor Cells. PLoS One 10, e0136405 (2015).
11. Tu, T. C., et al. A Chemokine Receptor, CXCR4, Which Is Regulated by Hypoxia-Inducible Factor 2alpha, Is Crucial for Functional Endothelial Progenitor Cells Migration to Ischemic Tissue and Wound Repair. Stem Cells Dev 25, 266-276 (2016).
12. Eibel, B., et al. VEGF gene therapy cooperatively recruits molecules from the immune system and stimulates cell homing and angiogenesis in refractory angina. Cytokine 91, 44-50 (2017).
13. Kim, H., Kim, S., Baek, S. H. & Kwon, S. M. Pivotal Cytoprotective Mediators and Promising Therapeutic Strategies for Endothelial Progenitor Cell-Based Cardiovascular Regeneration. Stem Cells Int 2016, U.S. Pat. No. 8,340,257 (2016).
14. Wei, G., et al. Hydroxysafflor yellow A promotes neovascularization and cardiac function recovery through HO-1/VEGF-A/SDF-1alpha cascade. Biomed Pharmacother 88, 409-420 (2017).
15. Qi, Y., et al. Inhaled NO contributes to lung repair in piglets with acute respiratory distress syndrome via increasing circulating endothelial progenitor cells. PLoS One 7, e33859 (2012).
16. Sobrino, T., et al. Temporal profile of molecular signatures associated with circulating endothelial progenitor cells in human ischemic stroke. J Neurosci Res 90, 1788-1793 (2012).
17. Paczkowska, E., et al. Increased circulating endothelial progenitor cells in patients with haemorrhagic and ischaemic stroke: the role of endothelin-1. J Neurol Sci 325, 90-99 (2013).
18. Turunen, A. J., et al. Matrix Metalloproteinase-9 and Graft Preservation Injury in Clinical Renal Transplantation. Transplant Proc 47, 2831-2835 (2015).
19. Hu, Q., et al. Therapeutic application of gene silencing MMP-9 in a middle cerebral artery occlusion-induced focal ischemia rat model. Exp Neurol 216, 35-46 (2009).
20. McKeown, S., et al. MMP expression and abnormal lung permeability are important determinants of outcome in IPF. Eur Respir J 33, 77-84 (2009).
21. Willems, S., et al. Multiplex protein profiling of bronchoalveolar lavage in idiopathic pulmonary fibrosis and hypersensitivity pneumonitis. Annals of thoracic medicine 8, 38-45 (2013).
22. Gukovsky, I., et al. A rat model reproducing key pathological responses of alcoholic chronic pancreatitis. Am J Physiol Gastrointest Liver Physiol 294, G68-79 (2008).
23. Cheung, P. Y., et al. Matrix metalloproteinase-2 contributes to ischemia-reperfusion injury in the heart. Circulation 101, 1833-1839 (2000).
24. Falk, V., et al. Regulation of matrix metalloproteinases and effect of MMP-inhibition in heart transplant related reperfusion injury. Eur J Cardiothorac Surg 22, 53-58 (2002).
25. Romanic, A. M., et al. Myocardial protection from ischemia/reperfusion injury by targeted deletion of matrix metalloproteinase-9. Cardiovasc Res 54, 549-558 (2002).
26. de Smet, B. J., et al. Metalloproteinase inhibition reduces constrictive arterial remodeling after balloon angioplasty: a study in the atherosclerotic Yucatan micropig. Circulation 101, 2962-2967 (2000).
27. George, S. J., et al., Surgical preparative injury and neointima formation increase MMP-9 expression and MMP-2 activation in human saphenous vein. Cardiovasc Res 33, 447-459 (1997).
28. Guan, W., et al. Acute treatment with candesartan reduces early injury after permanent middle cerebral artery occlusion. Translational stroke research 2, 179-185 (2011).
29. Teraa, M., et al. Bone marrow alterations and lower endothelial progenitor cell numbers in critical limb ischemia patients. PLoS One 8, e55592 (2013).
30. Levesque, J. P., Hendy, J., Takamatsu, Y., Simmons, P. J. & Bendall, L. J. Disruption of the CXCR4/CXCL12 chemotactic interaction during hematopoietic stem cell mobilization induced by GCSF or cyclophosphamide. J Clin Invest 111, 187-196 (2003).

### REFERENCES—EXAMPLE 1

1. DeLeve L D, et al. VEGF-sdf1 recruitment of CXCR7+ bone marrow progenitors of liver sinusoidal endothelial cells promotes rat liver regeneration. American Journal of Physiology-Gastrointestinal and Liver Physiology 2016.
2. Wang L, et al., Hepatic vascular endothelial growth factor regulates recruitment of rat liver sinusoidal endothelial cell progenitor cells. Gastroenterology 2012; 143:1555-1563.
3. Wang L, et al. Liver Sinusoidal Endothelial Cell Progenitor Cells Promote Liver Regeneration in Rats. Journal of Clinical Investigation 2012; 122:1567-1573.
4. Ding B-S, et al. Inductive angiocrine signals from sinusoidal endothelium are required for liver regeneration. Nature 2010; 468:310-315.
5. Maher J J. Cell-specific expression of hepatocyte growth factor in liver: Upregulation in sinusoidal endothelial cells after carbon tetrachloride. Journal of Clinical Investigation 1993; 91:2244-2252.
6. LeCouter J, et al. Angiogenesis-independent endothelial protection of liver: role of VEGFR-1. Science 2003; 299:890-893.
7. Ding B S, Cao Z, Lis R, Nolan D J, Guo P, Simons M, Penfold M E, et al. Divergent angiocrine signals from vascular niche balance liver regeneration and fibrosis. Nature 2014; 505:97-102.
8. Harb R, et al. Bone marrow progenitor cells repair rat hepatic sinusoidal endothelial cells after liver injury. Gastroenterology 2009; 137:704-712.
9. Xie G, L. W, X. W, L. W, DeLeve L D. Isolation of periportal, mid-lobular and centrilobular rat liver sinusoidal endothelial cells enables study of zonated drug toxicity. Am J Physiol-Gastrointest Liver Physiol 2010; 299:G1204-1210.
10. Upadhya G A, et al. Evidence of a role for matrix metalloproteinases in cold preservation injury of the liver in humans and in the rat. Hepatology 1997; 26:922-928.
11. DeLeve L D, et al. Prevention of sinusoidal obstruction syndrome (hepatic venoocclusive disease) in the rat by matrix metalloproteinase inhibitors. Gastroenterology 2003; 125:882-890.
12. Ito Y, Abril E R, et al. Inhibition of matrix metalloproteinases minimizes hepatic microvascular injury in response to acetaminophen in mice. Toxicological Sciences 2005; 83:190-196.
13. Hamada T, et al. Metalloproteinase-9 deficiency protects against hepatic ischemia/reperfusion injury. Hepatology 2008; 47:186-198.
14. Moore C, Shen X D, Gao F, Busuttil R W, Coito A J. Fibronectin-alpha4beta1 integrin interactions regulate metalloproteinase-9 expression in steatotic liver ischemia and reperfusion injury. The American journal of pathology 2007; 170:567-577.
15. Hori T, Uemoto S, Chen F, Ann-Baine M T, Gardner L B, Hata T, Kuribayashi K, et al. Effect of cold ischemia/reperfusion injury and/or shear stress with portal hypertension on the expression of matrix metalloproteinase-9. Ann Gastroenterol 2012; 25:345-351.
16. Rundhaug J E. Matrix metalloproteinases and angiogenesis. J Cell Mol Med 2005; 9:267-285.
17. Cai H, et al. Hypoxia Response Element-Regulated MMP-9 Promotes Neurological Recovery via Glial Scar Degradation and Angiogenesis in Delayed Stroke. Mol Ther 2017; 25:1448-1459.
18. Webb A H, Gao B T, Goldsmith Z K, Irvine A S, Saleh N, Lee R P, Lendermon J B, et al. Inhibition of MMP-2 and MMP-9 decreases cellular migration, and angiogenesis in in vitro models of retinoblastoma. BMC Cancer 2017; 17:434.
19. Lee S, Jilani S M, et al. Processing of VEGF-A by matrix metalloproteinases regulates bioavailability and vascular patterning in tumors. The Journal of Cell Biology 2005; 169:681-691.
20. Klein G, Schmal O, Aicher W K. Matrix metalloproteinases in stem cell mobilization. Matrix Biology 2015; 44-46:175-183.
21. Levesque J P, Hendy J, Takamatsu Y, Simmons P J, Bendall L J. Disruption of the CXCR4/CXCL12 chemotactic interaction during hematopoietic stem cell mobilization induced by GCSF or cyclophosphamide. The Journal of clinical investigation 2003; 111:187-196.
22. DeLeve L D, Wang X, McCuskey M K, McCuskey R S. Rat liver endothelial cells isolated by anti-CD31 immunomagnetic sorting lack fenestrae and sieve plates. American Journal of Physiology—Gastrointestinal and Liver Physiology 2006; 291:G1187-1189.
23. Steffan A M, et al. Phagocytosis, an unrecognized property of murine endothelial liver cells. Hepatology 1986; 6:830-836.
24. Golub L M, Ramamurthy N S, McNamara T F, Greenwald R A, Rifkin B R. Tetracyclines inhibit connective tissue breakdown: new therapeutic implications for an old family of drugs. Crit Rev Oral Biol Med 1991; 2:297-321.
25. Greenwald R A, Moak S A, Ramamurthy N S, Golub L M. Tetracyclines suppress matrix metalloproteinase activity in adjuvant arthritis and in combination with flurbiprofen, ameliorate bone damage. J Rheumatol 1992; 19:927-938.
26. Wack K E, et al. Sinusoidal ultrastructure evaluated during the revascularization of regenerating rat liver. Hepatology 2001; 33:363-378.
27. Kim T H, Mars W M, Stolz D B, Michalopoulos G K. Expression and activation of pro-MMP-2 and pro-MMP-9 during rat liver regeneration. Hepatology 2000; 31:75-82.
28. Keyt B A, et al. The Carboxyl-terminal Domain(111165) of Vascular Endothelial Growth Factor Is Critical for Its Mitogenic Potency. Journal of Biological Chemistry 1996; 271:7788-7795.
29. Cursio R, Mari B, Louis K, Rostagno P, Saint-Paul M C, Giudicelli J, Bottero V, et al. Rat liver injury after normothermic ischemia is prevented by a phosphinic matrix metalloproteinase inhibitor. FASEB Journal 2002; 16:93-95.
30. Ma Z Y, et al. Inhibition of Matrix Metalloproteinase-9 Attenuates Acute Small-for-Size Liver Graft Injury in Rats. American Journal of Transplantation 2010; 10:784-795.
31. Cheung P Y, Sawicki G, Wozniak M, Wang W, Radomski M W, Schulz R.

Matrix metalloproteinase-2 contributes to ischemia-reperfusion injury in the heart. Circulation 2000; 101:1833-1839.
32. Falk V, et al. Regulation of matrix metalloproteinases and effect of MMP-inhibition in heart transplant related reperfusion injury. Eur J Cardiothorac Surg 2002; 22:53-58.
33. Hu Q, et al. Therapeutic application of gene silencing MMP-9 in a middle cerebral artery occlusion-induced focal ischemia rat model. Exp Neurol 2009; 216:35-46.
34. Guan W, Kozak A, El-Remessy A B, Johnson M H, Pillai B A, Fagan S C. Acute treatment with candesartan reduces early injury after permanent middle cerebral artery occlusion. Transl Stroke Res 2011; 2:179-185.
35. McKeown S, Richter A G, O'Kane C, McAuley D F, Thickett D R. MMP expression and abnormal lung permeability are important determinants of outcome in IPF. Eur Respir J 2009; 33:77-84.
36. Willems S, Verleden S E, Vanaudenaerde B M, Wynants M, Dooms C, Yserbyt J, Somers J, et al. Multiplex protein profiling of bronchoalveolar lavage in idiopathic pulmonary fibrosis and hypersensitivity pneumonitis. Ann Thorac Med 2013; 8:38-45.
37. Turunen A J, et al. Matrix Metalloproteinase-9 and Graft Preservation Injury in Clinical Renal Transplantation. Transplant Proc 2015; 47:2831-2835.
38. Gukovsky I, Lugea A, Shahsahebi M, Cheng J H, Hong P P, Jung Y J, Deng Q G, et al. A rat model reproducing key pathological responses of alcoholic chronic pancreatitis. Am J Physiol Gastrointest Liver Physiol 2008; 294:G68-79.
39. George S J, Zaltsman A B, Newby A C. Surgical preparative injury and neointima formation increase MMP-9 expression and MMP-2 activation in human saphenous vein. Cardiovasc Res 1997; 33:447-459.
40. Tu T C, Nagano M, Yamashita T, Hamada H, Ohneda K, Kimura K, Ohneda O. A Chemokine Receptor, CXCR4, Which Is Regulated by Hypoxia-Inducible Factor 2alpha, Is Crucial for Functional Endothelial Progenitor Cells Migration to Ischemic Tissue and Wound Repair. Stem Cells Dev 2016; 25:266-276.
41. Kim H, Kim S, Baek S H, Kwon S M. Pivotal Cytoprotective Mediators and Promising Therapeutic Strategies for Endothelial Progenitor Cell-Based Cardiovascular Regeneration. Stem Cells Int 2016; 2016:8340257.
42. Sobrino T, Perez-Mato M, Brea D, Rodriguez-Yanez M, Blanco M, Castillo J. Temporal profile of molecular signatures associated with circulating endothelial progenitor cells in human ischemic stroke. J Neurosci Res 2012; 90:1788-1793.
43. Paczkowska E, et al. Increased circulating endothelial progenitor cells in patients with haemorrhagic and ischaemic stroke: the role of endothelin-1. J Neurol Sci 2013; 325:90-99.
44. Paczkowska E, et al. Evidence for proangiogenic cellular and humoral systemic response in patients with acute onset of spinal cord injury. J Spinal Cord Med 2015; 38:729-744.
45. Qi Y, et al. Inhaled NO contributes to lung repair in piglets with acute respiratory distress syndrome via increasing circulating endothelial progenitor cells. PLoS One 2012; 7:e33859.
46. Bo C J, et al. Effects of ischemic preconditioning in the late phase on homing of endothelial progenitor cells in renal ischemia/reperfusion injury. Transplant Proc 2013; 45:511-516.
47. Kim B J, et al. Synergistic vasculogenic effects of AMD3100 and stromal-cell-derived factor-1alpha in vasa nervorum of the sciatic nerve of mice with diabetic peripheral neuropathy. Cell Tissue Res 2013; 354:395-407.
48. Kawakami Y, et al. SDF-1/CXCR4 axis in Tie2-lineage cells including endothelial progenitor cells contributes to bone fracture healing. J Bone Miner Res 2015; 30:95-105.
49. Herrmann M, Verrier S, Alini M. Strategies to Stimulate Mobilization and Homing of Endogenous Stem and Progenitor Cells for Bone Tissue Repair. Front Bioeng Biotechnol 2015; 3:79.
50. Chiang K H, et al. Statins, HMG-CoA Reductase Inhibitors, Improve Neovascularization by Increasing the Expression Density of CXCR4 in Endothelial Progenitor Cells. PLoS One 2015; 10:e0136405.

A number of embodiments of the invention have been described. Nevertheless, it can be understood that various modifications may be made without departing from the spirit and scope of the invention. Accordingly, other embodiments are within the scope of the following claims.

1: A method for:
(i) enhancing or accelerating a liver regeneration,
(ii) enhancing or accelerating repair of an organ or tissue,
(iii) reducing the extent of or abolishing ischemia-reperfusion injury in an organ or tissue, or
(iv) enhancing bone marrow endothelial progenitor cell mobilization and engraftment in an organ or tissue,
wherein optionally the bone marrow progenitor cell comprises a bone marrow progenitor of liver sinusoidal endothelial cell;
comprising:
(a) (i) administering to an individual in need thereof, or to an organ, in need thereof if the organ is a transplant organ or a cadaver or donor organ or tissue intended for transplant, a compound or composition capable of inhibiting or decreasing the expression or activity of a matrix metalloproteinase (MMP) in an organ-specific or an organ-selective manner, or
(ii) administering to an organ of an individual in need thereof, or to the organ or tissue in need thereof if the organ or tissue is a transplant organ or tissue or a cadaver or donor organ or tissue intended for transplant or study, a compound or composition capable of inhibiting or decreasing the expression or activity of a matrix metalloproteinase (MMP);
or
(b)
(1) providing a compound or composition capable of inhibiting or decreasing the expression or activity of a matrix metalloproteinase (MMP) in a liver-specific or liver-selective manner,
(2) administering the compound or composition to the individual in need thereof if the compound or composition acts in an organ-specific manner, or contacting the organ or tissue with the compound or composition,
wherein optionally the organ is a transplant organ or a cadaver or donor organ or tissue intended for transplant, and optionally the compound or composition is administered to the organ before removal of the organ from the cadaver or donor,
(i) enhancing or accelerating a liver regeneration,
(ii) enhancing or accelerating repair of an organ or tissue,
(iii) reducing the extent of or abolishing ischemia-reperfusion injury in an organ or tissue,
(iv) enhancing bone marrow endothelial progenitor cell mobilization and engraftment in an organ or tissue,

2: The method of claim 1, wherein:
(a) the compound or composition capable of inhibiting or decreasing the expression or activity of the MMP protein, transcript and/or gene, optionally in a liver-selective manner, is or comprises:
(1) a nucleic acid, and optionally the nucleic acid is an inhibitory nucleic acid comprising: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme capable of capable of inhibiting or decreasing the expression or activity of the MMP protein, transcript and/or gene,
(2) a peptide or polypeptide, wherein optionally the polypeptide is or comprises an antibody or fragment thereof or equivalent thereof, capable of specifically binding the MMP, and is capable of inhibiting or decreasing the activity of the MMP enzyme, transcript and/or gene, or
(3) a small molecule, lipid, saccharide, nucleic acid or polysaccharide capable of inhibiting or decreasing the activity of the MMP enzyme, transcript and/or gene,
wherein optionally the small molecule comprises prinomastat, marimastat, batimastat, cipemastat, ilomastat (also known as galardin), rebimastat, tanomastat or any combination thereof,
(b) the compound or composition is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally;
wherein optionally the formulation or pharmaceutical composition is contained in or carried in a nanoparticle, a particle, a micelle or a liposome or lipoplex, a polymersome, a polyplex or a dendrimer; or
(c) the compound or composition, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant.

3: The method of claim 1, wherein the nucleic acid capable of inhibiting or decreasing the expression or activity of the MMP enzyme, transcript and/or gene comprises or is contained in a nucleic acid construct or a chimeric or a recombinant nucleic acid, or an expression cassette, vector, plasmid, phagemid or artificial chromosome, optionally stably integrated into the cell's chromosome, or optionally stably episomally expressed, and optionally the cell is a liver cell or a cell in a liver tissue or organ.

4: The method of claim 1, wherein the cell or the liver cell is a mammalian cell, wherein optionally the mammalian cell is an animal or a human cell, or a brain, a lung, a pancreas, a kidney, muscle, bone, skin, trachea, arterial or venous blood vessels, intestine, or a heart cell.

5: A kit comprising a compound or composition or a formulation or a pharmaceutical composition of claim 1, and optionally comprising instructions on practicing a method of any one of the preceding claims.

6-8. (canceled)

9: A method for:
(i) enhancing an infused or exogenous progenitor cell, optionally a bone marrow endothelial progenitor cell, engraftment in an organ or a tissue, wherein optionally the organ or tissue is a heart, kidney, brain, muscle, bone, skin, trachea, arterial or venous blood vessels, intestine, spinal cord, lung, or a liver, by preventing or inhibiting proteolytic cleavage of a sdf1 in the organ or the tissue, thereby preserving the chemoattractant effect of the sdf1 and also preserving a bone marrow sdf1, thereby reducing release of an endogenous progenitor cell, optionally an endogenous bone marrow endothelial progenitor cell, from the bone marrow, to decrease competition for engraftment of the infused or exogenous progenitor cell by the endogenous progenitor cell,
(ii) reducing release of endogenous progenitor cells, optionally sprocs, from the bone marrow, and/or
(iii) preventing or inhibiting proteolytic cleavage of the sdf1 systemically;
comprising:
(a) administering to an individual in need thereof a compound or composition capable of inhibiting or decreasing the expression or activity of a matrix metalloproteinase (MMP) in a systemic manner, or treating the organ or tissue with the compound or composition capable of inhibiting or decreasing the expression or activity of the MMP, and optionally the treatment is in a perfusion bath; or
(b) (1) providing a compound or composition capable of inhibiting or decreasing the expression or activity of a matrix metalloproteinase (MMP) in a systemic manner,
wherein optionally the matrix metalloproteinase (MMP) is a matrix metalloproteinase-9 (MMP-9), and optionally the MMP inhibition is MMP-9 specific,
and optionally the MMP is MMP-1, MMP-2, MMP-3, MMP-11, or MMP-13, and optionally the MMP inhibition is MMP-1, MMP-2, MMP-3, MMP-11, or MMP-13 specific; and
(2) administering the compound or composition to the individual in need thereof,
thereby
(i) enhancing infused or exogenous progenitor cell, optionally bone marrow endothelial progenitor cell, engraftment in an organ or a tissue, wherein optionally the organ or tissue is a heart, kidney, brain, spinal cord, lung, or a liver, by preventing or inhibiting proteolytic cleavage of a sdf1 in the organ or the tissue, thereby preserving the chemoattractant effect of the sdf1 and also preserving a bone marrow sdf1, thereby reducing release of an endogenous progenitor cell, optionally an endogenous bone marrow endothelial progenitor cell, from the bone marrow, to decrease competition for engraftment of the infused or exogenous progenitor cell by the endogenous progenitor cell,
(ii) reducing release of endogenous progenitor cells, optionally sprocs, from the bone marrow, and/or
(iii) preventing or inhibiting proteolytic cleavage of the sdf1 systemically.

10: The method of claim 9, wherein:
(a) the compound or composition capable of inhibiting or decreasing the expression or activity of the MMP protein, transcript and/or gene, optionally in an organ-selective manner, is or comprises:
(1) a nucleic acid, and optionally the nucleic acid is an inhibitory nucleic acid comprising: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme capable of capable of inhibiting or decreasing the expression or activity of the MMP protein, transcript and/or gene,
(2) a peptide or polypeptide, wherein optionally the polypeptide is or comprises an antibody or fragment thereof or equivalent thereof, capable of specifically binding the MMP, and is capable of inhibiting or decreasing the activity of the MMP enzyme, transcript and/or gene, or
(3) a small molecule, lipid, saccharide, nucleic acid or polysaccharide capable of inhibiting or decreasing the activity of the MMP enzyme, transcript and/or gene,
wherein optionally the small molecule comprises prinomastat, marimastat, batimastat, cipemastat, ilomastat (also known as galardin), rebimastat, tanomastat or any combination thereof,
(b) the compound or composition is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally;
wherein optionally the formulation or pharmaceutical composition is contained in or carried in a nanoparticle, a particle, a micelle or a liposome or lipoplex, a polymersome, a polyplex or a dendrimer; or
(c) the compound or composition, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant.

11: The method of any one of the preceding claims, wherein the nucleic acid capable of inhibiting or decreasing the expression or activity of the MMP enzyme, transcript and/or gene comprises or is contained in a nucleic acid construct or a chimeric or a recombinant nucleic acid, or an expression cassette, vector, plasmid, phagemid or artificial chromosome, optionally stably integrated into the cell's chromosome, or optionally stably episomally expressed, and optionally the cell is a liver cell or a cell in a liver tissue or organ.

12: The method of any one of the preceding claims, wherein the cell or the liver cell is a mammalian cell, wherein optionally the mammalian cell is an animal or a human cell, or a brain, a lung, a pancreas, a kidney or a heart cell.

13-14. (canceled)

15: The method of claim 1, wherein the enhancing or accelerating a liver regeneration comprises enhancing or accelerating liver regeneration after tissue injury, toxic liver injury, acute liver failure, liver transplantation, living-donor related liver transplantation, or liver resection.

16: The method of claim 1, wherein the enhancing or accelerating repair of an organ or tissue comprises enhancing or accelerating liver repair.

17: The method of claim 16, wherein the enhancing or accelerating liver repair comprises enhancing or accelerating repair of a liver after a trauma, an injury or an infection.

18: The method of claim 1, wherein the enhancing or accelerating repair of an organ or tissue comprises enhancing or accelerating repair of an ischemia-reperfusion injury, a heart attack or a stroke.

19: The method of claim 1, wherein the reducing the extent of or abolishing ischemia-reperfusion injury in an organ or tissue comprises reducing the extent of or abolishing ischemia-reperfusion injury in a normal liver or a fatty liver, a brain, lung, pancreas, kidney, muscle, bone, skin, trachea, arterial or venous blood vessels, intestine, spinal cord, nerve or a brain or a heart, or in a cadaver or a donor organ or tissue, or in a liver or a transplant liver or a heart or a lung, pancreas, kidney, muscle, bone, skin, trachea, arterial or venous blood vessels, intestine, spinal cord, nerve or a brain or transplant heart.

20: The method of claim 1, wherein the enhancing bone marrow endothelial progenitor cell mobilization and engraftment in an organ or tissue comprises enhancing bone marrow endothelial progenitor cell mobilization and engraftment in a heart, a brain or a liver, or enhancing chemoattraction of bone marrow progenitor cells to an organ, optionally a heart, a brain, lung, pancreas, kidney, muscle, bone, skin, trachea, arterial or venous blood vessels, intestine, spinal cord, nerve or a brain or a liver.

21: The method of claim 1, wherein administering to an organ of an individual in need thereof comprises administering to a liver, a heart, muscle, pancreas, bone, skin, trachea, arterial or venous blood vessels, intestine, spinal cord, nerve or a brain.

22: The method of claim 1, wherein the matrix metalloproteinase (MMP) is a matrix metalloproteinase-9 (MMP-9).

23: The method of claim 22, wherein the MMP inhibition is MMP-9 specific.

24: The method of claim 22, wherein the MMP is MMP-1, MMP-2, MMP-3, MMP-11, or MMP-13, and optionally the MMP inhibition is MMP-1, MMP-2, MMP-3, MMP-11, or MMP-13 specific.

25: The method of claim 1, wherein the compound or composition acts in an organ-specific or an organ-selective manner if the compound or composition is administered systemically, or the organ is treated with the compound or composition capable of inhibiting or decreasing the expression or activity of the MMP in a perfusion bath.
